(19) United States
(12) Patent Application Publication
(10) Pub. No.: US 2021/0128792 A1
(43) Pub. Date: May 6, 2021

(54) ELECTROSPUN MATRIX AND METHOD

(71) Applicant: AUCKLAND UNISERVICES LTD, Auckland (NZ)

(72) Inventors: Peter Roderick DUNBAR, Auckland (NZ); Vaughan J. FEISST, Auckland (NZ); Thomas KERR-PHILIPS, Auckland (NZ); Michelle Barbara LOCKE, Auckland (NZ); Jenny MALMSTROM-PENDRED, Auckland (NZ); Jadranka TRAVAS-SEJDIC, Auckland (NZ); Bryon WRIGHT, Auckland (NZ)

(21) Appl. No.: 16/472,305

(22) PCT Filed: Dec. 28, 2017
(86) PCT No.: PCT/NZ2017/050177

§ 371 Date: Jun. 21, 2019

(30) Foreign Application Priority Data
Dec. 28, 2016 (GB) 1622340.6

(51) Int. Cl. A61L 27/60; A61L 27/26; A61L 27/24

(52) U.S. Cl. CPC A61L 27/60; A61L 2400/12; A61L 27/24; A61L 27/26

(57) ABSTRACT

The present disclosure relates a biocompatible biodegradable polymer matrix which serves as a template for the growth of differentiated skin tissue comprising a dermis and an epidermis, the combination of the matrix and the differentiated skin, the method of preparing the same and the tissue obtainable from said method. The disclosure also extends to use of the differentiated skin in treatment. Thus, there is provided a matrix of electrospun fibres for growing differentiated skin tissue prepared by electrospinning solution of a biocompatible biodegradable polymer, wherein the electrospun fibres are about 0.3 μm to about 5 μm in diameter for example 1 to 5 μm, such as 1, 1.5, 2, 2.5, 3, 3.5, 4, or 4.5 μm

The present disclosure relates a biocompatible biodegradable polymer matrix which serves as a template for the growth of differentiated skin tissue comprising a dermis and an epidermis, the combination of the matrix and the differentiated skin, the method of preparing the same and the tissue obtainable from said method. The disclosure also extends to use of the differentiated skin in treatment.

### BACKGROUND

WO2016/209089, Dunbar et al, discloses a device for culturing fibroblasts cells and keratinocytes, to form differentiated skin tissue. A scaffold, template or matrix is typically employed to support the growth and differentiation of the skin cells. In Dunbar et al, the cell culture device comprises a frame which holds a matrix (also referred to as a substrate). The matrix can be moved between 2 orientations. In the first orientation the cells are on the surface of the matrix and not in contact with a gas permeable membrane in the device. Once the cells have had time to attach, typically after about 24-48 hours, then the device is inverted to the second orientation such that the cells on the surface of the matrix are now in contact with, or in very close proximity to the gas permeable membrane. This proximity is what induces the keratinocytes to differentiate and form a stratified epidermis. Prior to the Dunbar et al device, epidermal stratification was achieved by growing the cells at an air-liquid interface. Thus, Dunbar device eliminates the need for an air-liquid interface by using a gas permeable interface instead.

Whilst the ability to change the orientation the platform holding the cells, during growth, is vitally important to the differentiation of the cells, the present inventors have now established that the nature of the matrix support employed has a significant impact on the growth and differentiation of the cells.

It is important the fibroblast cells are able migrate into and through the matrix to form a dermis. However, if the pores of the matrix are too large then the matrix does not adequately support a “layer” of keratinocytes in contact/communication with the gas permeable membrane, which may prevent the epidermis from forming adequately.

In addition, naturally generated human skin has Rete ridges which are contours at the dermal-epidermal interface that provide resistance to shear force. In contrast, skin grown on currently available matrices lack Rete ridges which makes the skin vulnerable to damage from shear forces during handling, application of the skin graft to the patient, and during subsequent procedures, for example covering with wound dressings. This lack of robustness is a significant disadvantage of synthetic skin products.

Thus, there is a need for an improved scaffold or matrix which can support the growth of skin cells and achieve proper epidermal stratification.

### SUMMARY OF INVENTION

The invention is summarised in the following paragraphs:
1. A matrix of electrospun fibres for growing differentiated skin tissue prepared by electrospinning a solution of a biocompatible biodegradable polymer, wherein the electrospun fibres are about 0.3 μm to about 5 μm in diameter, such as 1, 1.5, 2, 2.5, 3, 3.5, 4, or 4.5 μm.
2. A matrix according to paragraph 1, wherein the biocompatible biodegradable polymer is selected from the group PLGA, PLA, PCL, PHBV, PDO, PGA, PLCL, PLLA-DLA, PEUU, cellulose-acetate, PEG-b-PLA, EVOH, PVA, PEO, PVP, blended PLA/PCL, gelatin-PVA, PCT/collagen, sodium aliginate/PEO, chitosan/PEO, chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan, PDLA/HA, PLLA/HA, gelatin/HA, gelatin/siloxane, PLLA/MWNTs/HA, PLGA/HA, dioxanone linear homopolymer (such as 100 dioxanone linear homopolymer) and combinations of two or more of the same.
3. A matrix according to paragraph 1 or 2 wherein the polymer is poly(lactic-co-glycolic acid) (PLGA).
4. A matrix according to any one of paragraphs 1 to 3, wherein the concentration of biocompatible biodegradable polymer is 26% to 40% w/v, for example 27, 28, 29, 30, 31, 32, 33, 34, 35, 36, 37, 38 or 39% w/v.
5. A matrix according to paragraph 4, wherein the concentration of biocompatible biodegradable polymer is 35% w/v.
6. A matrix according to any one of paragraph 2 to 5, wherein the ratio of poly-lactic acid to poly-glycolic acid in the PLGA is in the range 90:10 to 50:50 respectively, such as 85:15, 80:20, 75:25, 70:30, 65:35 or 60:40.
7. A matrix according to paragraph 6, wherein the ratio of poly-lactic acid to poly-glycolic acid is 75:25 to 65:35, respectively, such as 65:35.
8. A matrix according to any one of paragraph 1 to 7 wherein the solvent comprises one or more independently selected from chloroform, ethanol, acetic acid HFIP, propan-2-ol, acetic acid, tetrahydrofuran, DMSO, DMF, ethyl acetate, 1,4-dioxane, formic acid and water.
9. A matrix according to any one of paragraph 1 to 8, wherein a solvent comprising tetrahydrofuran is employed with the biocompatible biodegradable polymer.
10. A matrix according to any one of paragraph 1 to 9, wherein a solvent comprising dimethylformamide is employed with the biocompatible biodegradable polymer.
11. A matrix according to any one of paragraph 1 to 10, wherein a solvent comprising DCM is employed with the biocompatible biodegradable polymer.
12. A matrix according to any one of paragraph 1 to 10, wherein a solvent which is a 1:1 mixture of tetrahydrofuran and dimethylformamide is employed with the biocompatible biodegradable polymer.
13. A matrix according to any one of paragraph 1 to 12, wherein the electrospinning was performed at temperature in the range of 25 to 35° C., such as 30° C.
14. A matrix according to any one of paragraph 1 to 13, wherein a needle employed in electrospinning was positioned with its tip 10 cms from a collecting plate (such as a stainless steel collecting plate).
15. A matrix according to any one of paragraph 1 to 14, wherein a textured plate, for example micropatterned, such as undulating or dimpled, was employed to collect the electrospun fibres.
16. A matrix according to any one of paragraph 1 to 15, wherein the electrospinning was performed at flow rate of 0.4 mL per hour or lower, for example 0.1 to 0.4 mL, such as 0.3, 0.25, 0.2, 0.15 or 0.1 ml per hour.
17. A matrix according to any one of paragraph 1 to 16, wherein the matrix has a thickness of 100 μm or less for example 10 to 100 μm, such as 10, 20, 30, 40, 50, 60, 70, 80, 90, 95 or 100 μm.
18. A matrix according to any one of paragraphs 1 to 17, wherein the matrix is coated with an extracellular matrix protein or peptide thereof, for example a synthetic peptide (for example to promote cell adhesion and/or differentiation, such as the amino acid sequence of collagen, laminin and other extracellular matrix proteins or a peptide fragment or fragments thereof).
19. A matrix according to paragraph 18, wherein the extracellular matrix protein is selected from the group consisting of collagen IV, collagen I, laminin and fibronectin, or a combination thereof, such as collagen IV.
20. A matrix according to any one of paragraphs 1 to 19, comprises pores suitable for allowing migration of fibroblasts, for example the pores are in the range 2 to 30 microns, such as 15 to 25 microns, in particular 15, 16, 17, 18, 19, 20, 21, 22, 23, 24 or 25 microns.
21. A section of skin tissue comprising a matrix defined in any one of paragraph 1 to 20, wherein cells capable of forming into a dermis have migrated into the matrix.
22. A section of skin tissue according to paragraph 21, wherein the cells are fibroblasts.
23. A section of skin tissue according to paragraph 21 or 22, wherein epidermal cells capable of differentiating into an epidermis are accumulated on an external face (such an “upper planar” face) of the matrix.
24. A section of skin according to paragraph 23, wherein the said external surface of the matrix was pre-coated with collagen, such as collagen IV before the addition of the epidermal cells, such as keratinocytes and fibroblasts, to the culture.
25. A section of skin tissue according to paragraph 23 or 25, wherein the epidermal cells capable of differentiating into an epidermis are keratinocytes.
26. A section of skin tissue according to any one of paragraph 22 to 24, wherein there is a differentiated epidermal layer.
27. A section of skin tissue according to any one of paragraph 22 to 26, wherein there is a dermis layer.
28. A section of skin tissue according to paragraph 27, wherein the matrix is contained within the dermis layer.
29. A section of skin tissue according to any one of paragraph 21 to 28, wherein the biocompatible biodegradable polymer making up the matrix, has started degrading.
30. A section of skin tissue according to any one of paragraph 21 to 29, where the skin tissue comprises synthetic Rete ridges.
31. A section of skin according to any one of paragraph 21 to 30, contained in a device for culture and/or transportation, for example said device comprising:
   a container comprising a first endwall (bottom), and at least one sidewall,
   a detachable second endwall (top) adapted to engage with the container to define a chamber, and
   a scaffold adapted to receive a matrix for cells to reside upon,
   wherein at least a part of at least one of the first endwall (bottom), the at least one sidewall, or the second endwall (top) comprises a gas permeable material or is adapted to engage with a gas permeable material and is perforated to allow gaseous exchange; and
   wherein the apparatus is configurable between (a) a first mode in which the matrix is not disposed in gaseous communication with a gas permeable material, and (b) a second mode in which the substrate is disposed in gaseous communication with a gas permeable material.
32. A section of skin according to any one of paragraph 21 to 31, wherein the skin is autologous to a patient.
33. An ex vivo method of generating a differentiated skin product defined in any one of claims 1 to 32 comprising the steps:
   i) taking an electrospun matrix spun from a biocompatible biodegradable polymer in an organic solvent, wherein the electrospun fibres of the matrix are about 0.3 μm to about 5 μm in diameter, for example 1 to 5 μm, such as 1, 1.5, 2, 2.5, 3, 3.5, 4, or 4.5 μm, and
   ii) adding a culture of epidermal cells capable of differentiating into an epidermis (such as keratinocytes) and fibroblasts, and
   iii) culturing said cells and matrix in a device or pot comprising a gas permeable layer for a first period (for example up to one month, for example 30 days, 28 days, 21 days, 14 days, 7 days, 5 days, 4 days, 3 days, 48 hours or 24 hours) wherein the matrix face with epidermal cells deposited thereof is orientated distal from the gas permeable layer, and
   iv) after the first culture period changing the orientation of the matrix, such that the face with the epidermal cells deposited thereon is proximal to the gas permeable layer.
34. A method according to paragraph 33, wherein the face of the matrix on which the epidermal cells are to be deposited is pre-coated to encourage adherence and/or differentiation of the epithelial cells.
35. A method according to paragraph 34, wherein the coating is an extracellular matrix protein or a peptide thereof, for example a synthetic peptide sequence (such as the amino acid sequence of collagen, laminin and other extracellular matrix proteins proteins or a peptide fragment or fragments thereof).
36. A method according to paragraph 35, wherein the extracellular matrix protein or peptide thereof is selected from the group consisting of: collagen IV, collagen I, laminin and fibronectin, and a combination thereof, such a collagen IV.
37. A method according to any one of paragraph 33 to 36, wherein the biocompatible biodegradable polymer is selected from the group PLGA, PLA, PCL, PHBV, PDO, PGA, PLCL, PLLA-DLA, PEUU, cellulose-acetate, PEG-b-PLA, EVOH, PVA, PEO, PVP, blended PLA/PCL, gelatin-PVA, PCT/collagen, sodium aliginate/PEO, chitosan/PEO, chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan, PDLA/HA, PLLA/HA, gelatin/HA, gelatin/siloxane, PLLA/MWNTs/HA, PLGA/HA, 100 dioxanone linear homopolyer and combinations of two or more of the same.
38. A method according to any one of paragraph 33 to 37, wherein the polymer is poly(lactic-co-glycolic acid) (PLGA).
39. A method according to any one of paragraph 33 to 38, which comprises the pre-step of electrospinning the matrix.
40. A method according to paragraph 39, wherein the concentration of biocompatible biodegradable polymer is 26% to 40% w/v, for example 27, 28, 29, 30, 31, 32, 33, 34, 35, 36, 37, 38 or 39% w/v.
41. A method for growing differentiated skin tissue according to paragraph 40, wherein the concentration of biocompatible biodegradable polymer is 35% w/v.
42. A method according to any one of paragraph 33 to 41, wherein the ratio of poly-lactic acid to poly-glycolic acid in the PLGA is in the range 90:10 to 50:50 respectively, such as 85:15, 80:20, 75:25, 70:30, 65:35 or 60:40.
43. A method according to paragraph 42, wherein the ratio of poly-lactic acid to poly-glycolic acid is 75:25 to 65:35, respectively, such as 65:35.
44. A method according to any one of paragraph 39 to 43, wherein the solvent comprises one or more independently selected from chloroform, ethanol, acetic acid, hexafluoroisopropanol, propan-2-ol, acetic acid, DMSO, DMF, ethyl acetate, 1,4-dioxane, dimethylacetamide, methyl ethyl ketone, formic acid and water.
45. A method according to any one of paragraphs 39 to 44, wherein a solvent comprising tetrahydrofuran is employed with the biocompatible biodegradable polymer.
46. A method according to any one of paragraphs 39 to 45, wherein a solvent comprising dimethylformamide is employed with the biocompatible biodegradable polymer.
47. A method according to any one of paragraphs 39 to 46, wherein a solvent comprising DCM is employed with the biocompatible biodegradable polymer.
48. A method according to any one of paragraphs 39 to 47, wherein a solvent which is a 1:1 mixture of tetrahydrofuran and dimethylformamide, is employed with the biocompatible biodegradable polymer.
49. A method according to any one of paragraphs 39 to 48, wherein the electrospinning is performed at temperature in the range of 25 to 35° C., such as 30° C.
50. A method according to any one of paragraphs 39 to 49, wherein a needle employed in the electrospinning is located with its tip 10 cms from a collecting plate (such as a stainless steel collecting plate).
51. A method according to any one of paragraphs 39 to 50, wherein a textured plate, for example micropatterned, such as undulating or dimpled, is employed to collect the fibres.
52. A method according to any one of paragraphs 39 to 51, wherein the electrospinning is performed at flow rate of 0.4 mL per hour or lower, for example 0.1 to 0.4 mL, such as 0.3, 0.25, 0.2, 0.15 or 0.1 ml per hour.
53. A method according to any one of paragraphs 33 to 52, wherein the matrix has a thickness of 100 μm or less, for example 10 to 100 μm, such as 10, 20, 30, 40, 50, 60, 70, 80, 90, 95 or 100 μm.
54. A method according to any one of paragraphs 33 to 53, wherein the skin tissue is cultured in
   a device for culturing cells or tissue, for example comprising:
   a container comprising a first endwall (bottom), and at least one sidewall,
   a detachable second endwall (top) adapted to engage with the container to define a chamber,
   a scaffold adapted to receive a matrix for cells to reside upon,
   wherein at least a part of at least one of the first endwall (bottom), the at least one sidewall, or the second endwall (top) comprises a gas permeable material or is adapted to engage with a gas permeable material and is perforated to allow gaseous exchange; and
   wherein the apparatus is configurable between (a) a first mode in which the matrix is not disposed in gaseous communication with a gas permeable material, and (b) a second mode in which the substrate is disposed in gaseous communication with a gas permeable material.
55. A method according to any one of paragraphs 33 to 54, wherein the skin tissue is transported to a patient's location, for example is transported in the device defined in paragraph 54.
56. A method according to paragraphs 55, wherein the skin tissue is recovered from the device by a healthcare professional, such as a surgeon, for example by excising the tissue from a scaffold in the device arranged to hold the matrix and tissue.
57. An ex vivo method of growing differentiated skin tissue comprising Rete ridges, wherein the method comprises electrospinning a biocompatible biodegradable polymer matrix with pores suitable for supporting differentiated skin tissue growth onto a micropatterned plate.
58. An ex vivo method of growing differentiated skin tissue comprising Rete ridges said method comprising the step of growing the skin cells on a matrix with a undulating or dimpled surface (for example formed by electrospraying the matrix fibres on a textured collecting plate).
59. A section of differentiated skin tissue obtained or obtainable from any one of paragraphs 33 to 58.
60. A section of skin according to any one of paragraphs 21 to 31 or 59 for use in treatment
61. A section of skin according to paragraph 60, wherein the treatment is for a condition or disease selected from the group consisting of: tissue damage; skin regeneration with nerves & organelles; wound healing, for example promoting/enhancing wound healing, including ulcers such as diabetic ulcers; burn healing; skin regeneration and repair; epidermolysis bulosa; to replace the cancerous skin tissue excised by surgery (such as sarcoma, melanoma or the like), to replace skin tissue excised to try contain infection with necrotising fasciitis; to enhance skin quality or appearance; prevention or remediation of skin disorders; diminishment or abolishment of scar tissues; breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring.
62. A section of skin according to paragraph 61, for use in the treatment of a surgical scar, an ulcer, damage caused by necrotizing fasciitis, anything that may require a skin graft, for example skin cancer surgery, mole removal, a cut or stab wound, a wound caused by a shearing force, a graze, an abrasion, a chemical burn, psoriasis, skin infections, bed sores or similar.
63. A method oftreatment comprising suturing a section of skin according to any one of paragraphs 21 to 31 or 60 to a patient in need thereof
64. A method of treatment according to paragraphs 63, wherein the treatment is for a condition or disease selected from the group consisting of: tissue damage; skin regeneration with nerves & organelles; wound healing, for example promoting/enhancing wound healing, including ulcers such as diabetic ulcers; burn healing; skin regeneration and repair; epidermolysis bulosa; enhance skin quality or appearance; prevention or remediation of skin disorders; diminishment or abolishment of scar tissues; breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring.
65. A method of treatment according to paragraph 63, for use in the treatment of a surgical scar, an ulcer, damage caused by necrotizing fasciitis, anything that may require a skin graft, for example skin cancer surgery, mole removal, a cut or stab wound, a wound caused by a shearing force, a graze, an abrasion, a chemical burn, psoriasis, skin infections, bed sores or similar.
66. Use of a section of skin according to any one of paragraphs 21 to 31 or 60 for the manufacture of a medicament for the treatment is for a condition or disease selected from the group consisting of: tissue damage; skin regeneration with nerves & organelles; wound healing, for example promoting/enhancing wound healing, including ulcers such as diabetic ulcers; burn healing; skin regeneration and repair; epidermolysis bulosa; enhance skin quality or appearance; prevention or remediation of skin disorders; diminishment or abolishment of scar tissues; breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring.
67. Use of a section of skin according to any one of paragraphs 21 to 31 or 60, for the manufacture of a medicament for the treatment of a surgical scar, an ulcer, damage caused by necrotizing fasciitis, anything that may require a skin graft, for example skin cancer surgery, mole removal, a cut or stab wound, a wound caused by a shearing force, a graze, an abrasion, a chemical burn, psoriasis, skin infections, bed sores or similar.
68. A collector plate, such as a stainless steel collector plate, for collecting a electrospun fibres of matrix for growing tissue, wherein the collector plate comprises a micropatterned surface, for example an undulating, a dimpled or a wave pattern.
69. A collector plate according to paragraph 68, wherein the plate comprises holes, such that sheet of electrospun fibres formed within the hole is of a lower density than the sheet of electrospun fibres formed on the surface of the collector plate.
70. A matrix produced by electrospinning fibres onto a collector plate according to paragraphs 68 or 69, for example wherein at least one planar surface of the matrix has a undulating or dimpled surface.
71. A method of enhancing a cultured skin tissue's resistance to shear force comprising growing the skin tissue on a matrix, wherein the matrix comprises a micropatterned surface, for example undulating, a dimpled or a wave pattern.
72. A method according to paragraph 71, wherein the matrix is produced by electrospinning fibres onto a collector plate according to any one of claims 68 to 70.
72. A method according to paragraph 71, wherein the matrix is defined in any one of paragraphs 1 to 20.

Accordingly, in one aspect there is provided matrix for growing differentiated skin tissue prepared by electrospinning a 26% to 40% w/v solution of a biodegradable polymer, such as poly(lactic-co-glycolic acid) (PLGA), dioxanone linear homopolyer (such as 100 dioxanone linear homopolyer), or a combination thereof in an organic solvent. The presently disclosed matrix prepared by electrospinning a 26% to 40% w/v solution of PLGA in an organic solvent has a porosity which allows separation of epidermal and dermal cells so that an epidermis forms on one surface of the matrix, such as the “top” of the matrix (referred to herein as the “upper planar face” of the matrix), and a dermis forms in and on the opposite surface of the matrix, such as “below” the matrix (referred to herein as the lower planar face of the matrix). Thus, for example the keratinocytes stay on “top” of the matrix to form an epidermis and the fibroblasts are able to migrate into the matrix to make a dermis. The result is that the skin formed has the synthetic matrix integrated between the dermis and epidermis formed by the fibroblasts and keratinocytes respectively.

In one embodiment, the concentration of PLGA is 27, 28, 29, 30, 31, 32, 33, 34, 35, 36, 37, 38 or 39% w/v.

In one embodiment, the concentration of PLGA is 35% w/v. Advantageously, the present inventors found that electrospinning with a 35% w/v solution resulted in an optimise fibre diameter and porosity but was easier to work with than higher concentrations of PLGA.

In one embodiment, the ratio of poly-lactic acid to poly-glycolic acid in the PLGA is in the range 90:10 to 60:40 respectively, such as 85:15, 80:20, 75:25, 70:30, or 65:35. An advantage of using PLGA is that changing the ratio of poly glycolic acid to poly lactic acid changes the rate of degradation. Thus, the skilled person can adjust the rate of degradation as desired by adjusting the ratio of poly glycolic acid to poly lactic acid.

In one embodiment, the ratio of poly-lactic acid to poly-glycolic acid is 75:25 to 65:35, respectively, such as 75:25. Advantageously, 75:25 PLGA is expected to degrade completely in 3 to 12 months, whereas a lower ratio such as 63:35 would degrade ata faster rate.

There are many polymers, such as PLGA which are biocompatible and biodegradable, which are suitable for forming the matrix. Materials that employed in sutures may be suitable for forming the matrix. The matrix provides structural integrity for the engineered skin tissue whilst it is being grown and also for some time after it has been grafted onto a patient. The matrix will degrade over a period of weeks to months, eventually being completely replaced by the patient's own matrix (cells). This eliminates the requirement to recover the matrix from the patient.

Matrix in the context of the present disclosure is a three dimensional structure, in particular woven, fibre and/or spun matrix and on and in which the cells adhere and grow. The matrix is a substrate or template around which the cells gather, adhere and/or grow.

In one embodiment matrix is provided as slice. Slice as employed herein refers to a three dimensional shape, for example which is suitable template for a section of skin to grow on, for example about 100 microns in depth and with a two planar substantially faces.

Planar face refers to an approximately flat 2D surface, it does not need to be perfectly flat but simply needs to support the growth or cells, for example that will differentiate into an epidermis. In one embodiment the upper planar face is micropatterned to provide textures that will ultimately increase the resistance to shear forces of the differentiated epidermis (i.e. to reduce it sliding over the dermis and instead retain the epidermis in place). This texture is a mirror-image of texture provided on the collecting plate when the matrix fibres are electrospun. This texture is undulating or dimpled. This is intended to generate cell growth in an irregular interface between the dermis and epidermis to simulate the protrusions known as Rete ridges in normal skin.

“Upper planar face” as employed herein is not necessarily a reference to an orientation of the matrix but instead refers to one of the two planar faces on which the keratinocytes deposit. These cells ultimately differentiate into the epidermis and become the upper (outermost) layer of skin. The upper planar face will generally be pre-coated to encourage the keratinocytes to adhere to it. The upper planar surface at the start of the culture will generally be remote from the gas permeable layer. For example, where the scaffold holding the matrix is horizontal and parallel with a gas permeable layer in the base of the device the upper planar surface will be upper most, i.e. directed to the lid. However, after a suitable period, for example 48 hours when the keratinocytes have adhered to the upper planar face then the substrate (matrix) will be rotated or moved to put the upper planar face in contact with or in proximity to the gas permeable layer. This will generally put the upper planar face on the underside of the substrate (matrix), and thus direct the upper planar face towards the base of the device. Thus, the orientation of the upper planar face changes through the process. However, in the designating upper planar face applies to the face regardless of its orientation.

Lower planar face as employed herein refers to the corresponding parallel face to the upper planar face. Lower is not necessarily a reference to the orientation of the substrate face.

In one embodiment, the collecting plate is textured, for example micropatterned, such as undulating or dimpled. The advantage of the micropatterning is that an uneven interface is formed between the dermis and epidermis when the matrix is used to grow skin tissue, thereby replicating naturally occurring Rete ridges between the dermis and epidermis. The presence of the Rete ridges helps to confer resistance against shear forces for the epidermis which makes the epidermis less likely to be displaced if a shear force is applied, for example from a wound dressing or from clothes rubbing on the skin graft. Displacement of the epidermis would likely result in the failure of the skin graft.

Rete ridges in the context of the present disclosure is simply an uneven interface between to the dermis and the epidermis, i.e. is a synthetic (ex vivo grown) simulation natural Rete ridges.

In one embodiment the matrix has a thickness of 100 μm or less, for example 10 to 100 μm, such as 20, 25, 30, 35, 40, 45, 50, 55, 60, 65, 70, 75, 80, 85, 90 or 95 μm. The present inventors have found that using a matrix with a thickness greater than 100 μm tends to increase the need for vascularisation of the skin tissue grown on the matrix in order for the graft to survive. Conversely, by keeping the matrix 100 μm thick or less, the keratinocytes can receive nutrients and remove waste by diffusion alone and not require a vasculature system for survival.

In embodiment the matrix is formed a particular shape, for example a tube, lobe or the like, such that the skin cells grow into a predefined 3D shape, which is required for the patient. In one embodiment the pore size (for example minimum and maximum pore size) of the matrix is in the range are in the range 2 to 30 microns, for example 15 to 25 microns, such as 15, 16, 17, 18, 19, 20, 21, 22, 23, 24 or 25 microns.

The pores in the matrix will not all be of uniform size. Thus, the pore size as employed herein will be the average pore size, which for example are in the range 4 to 25 microns (4, 5, 6, 7, 8, 9, 10, 11, 12, 13, 14, 15, 16, 17, 18, 19, 20, 21, 22, 23, 24 or 25 microns), for example 10 to 25 microns, such as 10, 11, 12, 13, 14, 15, 16, 17, 18, 19, 20, 21, 22, 23, 24 or 25 microns, in particular 15 to 25 microns. In one embodiment the average is the mean pore size, for example the mean flow pore size (also known a capillary flow porometry). In one embodiment the average is the median pore size. In one embodiment the average is the modal pore size.

The factor that influences pore size most significantly is fibre diameter. The larger the fibre diameter the

Using porometry one of the most critical values in the largest pore size because this often determines important characteristics of the material. One measurement of the largest pore size in the first bubble point value (FBP). In one embodiment the pore size given herein is the first bubble point value.

Capillary flow porometry, also known as porometry, is a characterization technique based on the displacement of a wetting liquid from the sample pores by applying a gas at increasing pressure.

It is widely used to measure minimum, maximum (or first bubble point) and mean flow pore sizes, and pore size distribution of the through pores in membranes, paper, filtration and ultrafiltration media, hollow fibers, ceramics, etc. FIG. 7 shows a typical curve for the analysis.

In capillary flow porometry an inert gas is used to displace a liquid, which is in the pores. The pressure required to empty the pore corresponds to the pressure necessary to evacuate the liquid from the most constricted part of the pore. This most constricted part is the most challenging one and it offers the highest resistance to remove the wetting liquid. This parameter is very relevant in filtration and similar applications since it is important to know the smallest.

This measured pressure permits obtaining the pore diameter, which is calculated by using the Young-Laplace formula P=4*γ*cos θ*/D in which D is the pore size diameter, P is the pressure measured, γ is the surface tension of the wetting liquid and θ is the contact angle of the wetting liquid with the sample. The surface tension γ is a measurable physical property and depends on the wetting liquid used. The contact angle θ depends on the interaction between the material and the wetting liquid. In capillary flow porometry, in opposition to mercury intrusion porosimetry, the wetting liquid enters spontaneously the pores of the sample ensuring a total wetting of the material, and therefore the contact angle of the wetting liquid with the sample is θ and the previous formula can be simplified as: P=4*γ/D.

Technical information on how to perform the analysis and the instrument is available at www.porometer.com. See also Agarwal et al, Neck-size distribution of through-pores in polymer membranes, Journal of Membrane Science 415-416 (2012) 608-615.

### Pressure Scan

This is the traditional approach, in which the pressure increases continuously at a constant rate, which can be modified depending the instrument and the user's requirements, and the gas flow through the sample is measured. Again, the number of data points acquired can be adjusted by the user. It is a fast and reproducible method that is generally recommended for quality control work and for samples with all pores identical. However it is important to take into account that when the samples present a complex structure and with a considerable amount of pores of different tortuosity it is possible that during the pressure scan pores with the same diameter but longer pore path are not emptied at the pressure corresponding to their diameter (if the scan is fast there is not time to allow the gas flow to displace the wetting liquid through the pore length). As consequence, the pores with longer pore length will be report smaller pore sizes than the actual ones.

### Pressure Step/Stability

Increase of the pressure with respect of time in steps, holding the actual pressure for a certain time before increasing it to the next value.

The pressure/step stability method represents an alternative for the pressure scan method which permits a more accurate measurement of the pore sizes. It takes into account the different tortuosity and pore length of pores with the same diameter. The acquisition of a data point is only carried out after holding the pressure at a constant value for a user-defined time and also only after the gas flow through the sample is stable, which is also defined by the user. This allows enough time for the gas flow to displace the wetting liquid in long and tortuous pores of the same diameter. Therefore, the pressure step/stability method is the most recommended one for research and development applications. Additionally, the pressure step/stability measuring principle allows measuring the true First Bubble Point (FBP), in opposition to the pressure scan method, which only permits calculation the FBP at the selected flow rates.

### Measured First Bubble Point (FBP)

The FBP is defined by the ASTM F-316-03 standard as the pressure at which the first continuous gas bubbles are detected. In practice FBP is associated as the largest or maximum pore size. The calculation of the FBP requires to select a certain minimum flow (e.g. 30, 50, 100 ml/min) and when it is achieved record the pressure. Then this pressure is used to calculate the FBP size.

The question is to select the minimum flow through the sample and the major disadvantage is that this minimum flow is different for every sample and it is not easy to determine a priori. If a certain minimum flow is selected for the calculation it is possible that the largest pore in the sample was already open for a while before that particular flow was determined. With the step/stability method it is possible to measure the true FBP. When applying a constant gas flow, before the opening of the largest flow the pressure increases in a linear way. At the moment that the gas flow passes through the sample via the largest pore the pressure increase drops and it is this particular pressure the one that corresponds to the FBP of the sample.

In one embodiment, the matrix is coated with an extracellular matrix protein or peptide thereof, such as a synthetic peptide.

In one embodiment, the extracellular matrix protein is selected from the group consisting of: collagen IV, collagen I, laminin and fibronectin, or a combination thereof. Advantageously, the presence of the coating produces a second cellular signal (the first signal being growing the skin tissue at an air-liquid or gas permeable interface), which enhances the proper stratification of the skin tissue.

In one embodiment the extracellular matrix protein coating is collagen IV. The advantage of collagen IV is that it was found to consistently produce the best epidermal stratification compared to collagen I, laminin and fibronectin.

In one aspect, there is provided a section of skin tissue comprising a matrix as defined above, wherein dermis feeder cells, such as fibroblasts, have migrated into the matrix.

In one embodiment, keratinocytes stay on an exterior face of the matrix, in particular the pre-coated surface.

In one embodiment, the keratinocytes have differentiated into an epidermis and the fibroblasts have formed a dermis.

In one embodiment, the differentiated epidermis is located on one surface of the matrix, in particular the “upper planar face” of the matrix.

In one embodiment, the matrix is coated with collagen before the addition of the epithelial cells, such as keratinocytes and fibroblasts, to the culture.

In one embodiment, the skin tissue comprises synthetic Rete ridges, for example as described elsewhere herein.

In one embodiment, the skin is autologous to a patient. The advantage of this is that autologous skin tissue would not be at risk of tissue rejection when grafting onto the patient.

In one embodiment, the skin tissue is contained in a device as described herein, in particular a device is described in WO2016/209089 the contents of which are herein incorporated by reference. Advantageously, the device allows the skin tissue to be cultured in solution when in the first mode (allowing cell migration, adherence and/or expansion) and then to be grown at the gas permeable interface in the second mode, wherein the skin tissue can differentiate and achieve epidermal stratification.

In one embodiment, wherein the skin tissue is transported to a patient's location in the device as defined above. Advantageously, the device is a sealed and sterile suitable for both growing the skin tissue and also for transporting the grown skin tissue thereafter. Accordingly, handling of the skin tissue is considerably minimised. Recovered of the tissue from the device by a healthcare professional, such as a surgeon, for example by excising the tissue from the matrix using a scalpel, in particular by the surgeon cutting a piece of skin of the desired shape and size from the matrix. Hence, there is no need for entire skin tissue to be first recovered from the device and then cut to the desired shape and size. Instead the desired piece of skin can be directly cut from the matrix. This further minimises handling and therefore reduces the risk of contamination and/or damage.

Tissue as employed herein refers to a group of similar or associated cells that work to together in a way suitable to perform a function, in particular when incorporated into an organism. Examples of tissue include skin, connective tissue, muscle and the like.

Synthetic tissue as employed herein refers to tissue grown ex vivo.

Synthetic in the context of peptides simply refers to peptides that have be synthesised using chemistry techniques (as a opposed to recombinant techniques).

Structured synthetic tissue as employed herein refers to tissue with distinct/differentiated components, for example a dermis and an epidermis.

Dermis as employed herein refers to the inner of skin cells beneath the epidermis. In the body the dermis is located between the epidermis and the subcutaneous tissue. The matrix of the present disclosure is located within the dermis after the differentiated skin tissue has been grown. Within the dermis as employed herein refer to where at least part of the matrix is within at least part of the dermis.

Fibroblasts are capable of forming the dermis. They are also feeder cells to the epidermal cells such as keratinocytes.

Epidermis is the outer layer of cells in skin tissue, which provides a barrier to infection and regulates the amount of water released by the body. The epidermis is composed of 4 or 5 layers depending on the region of skin being considered. Those layers in descending order are: i) Cornified Layer (stratum corneum). Composed of 10 to 30 layers of polyhedral, anucleated corneocytes (final step of keratinocyte differentiation), with the palms and soles having the most layers. Corneocytes are surrounded by a protein envelope (cornified envelope proteins), filled with water-retaining keratin proteins, attached together through corneodesmosomes and surrounded in the extracellular space by stacked layers of lipids. Most of the barrier functions of the epidermis localize to this layer. ii) Clear/translucent layer (stratum lucidum, only in palms and soles) The skin found in the palms and soles is known as “thick skin” because it has 5 epidermal layers instead of 4. Granular Layer (stratum granulosum) Keratinocytes lose their nuclei and their cytoplasm appears granular. Lipids, contained into those keratinocytes within lamellar bodies, are released into the extracellular space through exocytosis to form a lipid barrier. Those polar lipids are then converted into non-polar lipids and arranged parallel to the cell surface. For example glycosphingolipids become ceramides and phospholipids become free fatty acids. iii) Spinous Layer (stratum spinosum). Keratinocytes become connected through desmosomes and start produce lamellar bodies, from within the Golgi, enriched in polar lipids, glycosphingolipids, free sterols, phospholipids and catabolic enzymes. Langerhans cells, immunologically active cells, are located in the middle of this layer. iv) Basal/Germinal layer (stratum basale/germinativum). Composed mainly of proliferating and non-proliferating keratinocytes, attached to the basement membrane by hemidesmosomes. Melanocytes are present, connected to numerous keratinocytes in this and other strata through dendrites. Merkel cells are also found in the stratum basale with large numbers in touch-sensitive sites such as the fingertips and lips. They are closely associated with cutaneous nerves and seem to be involved in light touch sensation. v) Malpighian layer (stratum malpighi) is both the stratum basale and stratum spinosum. The epidermis is separated from the dermis, its underlying tissue, by a basement membrane. The epidermis of the present disclosure may comprise one or more of said layers.

Epidermal cells as employed herein refers to cells capable of differentiating into an epidermis, such as keratinocytes.

First culture period as employed herein refers to the period of culture when the keratinocytes adhere to the upper planar face. One or more cells types may be added to the culture one or more times during the first culture period.

Second culture period as employed herein refers to the period of culture wherein the keratinocytes adhered to the upper planar face are put into contact with or in proximity to the gas permeable layer. One or more cells types may be added to the culture one or more times during the second culture period.

In the present disclosure “adding a culture of epidermal cells capable of differentiating into an epidermis . . . and fibroblasts” as employed herein includes wherein: fibroblasts are added first and cultured for a period followed by the addition of epidermal cells (or followed by addition of a combination of epidermal cells and fibroblasts); epidermal cells are added first and cultured for a period followed by the addition of fibroblasts (or followed by a combination of epidermal cells and fibroblasts); and a combination of epidermal cells and fibroblasts are added at essentially the same.

At essentially the same time as employed herein refers to where there is no culturing period between the addition of the relevant cells.

Cell populations employed in the present disclosure, need to be sufficiently pure to be fit for purpose. Thus other cells populations may also be present.

Further cells may be added to the culture at any time, as required.

Fibroblasts may be cultured in a pre-step, for example in the absence of the matrix, in a cell culture device with or without a gas permeable membrane, to increase the numbers.

In one aspect, there is provided a method of treatment, comprising:
a) providing a skin tissue according to as described above,
b) recovering under sterile conditions the tissue from the matrix, and
c) applying the tissue to a patient in need of treatment.

In one aspect, there is provided a collector plate, such as a stainless steel collector plate, for collecting a sheet of electrospun fibres, wherein the collector plate comprises a micropatterned surface, for example an undulating, a dimpled or a wave pattern, in particular for collecting a electrospun fibres of a matrix, in particular a matrix template for use in differentiated skin culture. The disclosure also extends to use of said plate to collect electrospun fibres of matrix for cell culture.

In one embodiment, the plate comprises holes, such that the sheet of electrospun fibres formed within the hole is of a lower density than the sheet of electrospun fibres formed on the surface of the collector plate. Advantageously, the present inventors have found that the lower density sheet of electrospun fibres forms a superior matrix for the growth of full thickness skin.

In one aspect, there is provided a synthetic matrix produced by electrospinning fibres onto a collector plate as defined above. The disclosure also extends to use of a matrix according to the disclosure as template for ex vivo culture of skin tissue, in particular differentiated skin tissue. In one aspect, there is provided a method of enhancing a cultured skin tissue's resistance to shear force comprising growing the skin tissue on a matrix, wherein the matrix comprises a micropatterned surface, for example undulating, a dimpled or a wave pattern.

In one embodiment, the matrix is produced by electrospinning fibres onto a collector plate as defined above.

In one embodiment, the matrix is a matrix as defined above.

### BRIEF DESCRIPTION OF FIGURES

FIG. 1 shows the electromicroscopy images of the fibre diameter of PLGA electrospun from a range of solution concentrations. a) 20% w/v, b) 25% w/v, c) 30% w/v, d) 35% w/v, and e) 40% w/v.

FIG. 2 shows the effect of fibre diameter and porosity on skin formation. Transverse sections of human skin cells grown on electrospun PLGA of produced from various concentrations, a) 20% w/v, b) 25% w/v, c) 35% w/v, d) 40% w/v. All sections are stained with pan-cytokeratin (light grey areas) to identify keratinocytes and DAPI (dots) to identify cell nuclei. 20% and 25% results in no dermis formed. 35 and 40% have a dermis formed underneath the epidermis, as indicated by the arrows.

FIG. 3 shows the effect of the protein coating on epidermal stratification. Transverse sections of human skin cells grown on uncoated electrospun PLGA and electrospun PLGA coated with human collagen IV. All sections are stained with pan-cytokeratin (light grey areas) to identify keratinocytes and DAPI (dots) to identify cell nuclei. Addition of the collagen IV coating induces epidermal stratification.

FIG. 4 shows images of a micropatterned electrospun PLGA sheet A) micropatterned, B) flat (unpatterned)

FIG. 5 shows images of skin grown on micropatterned electrospun PLGA vs unpatterned electrospun PLGA. Transverse section of human skin cells grown on micropatterned electrospun PLGA (left image) and unpatterned (flat) electrospun PLGA (right image) stained for vimentin (light grey regions between dashed line) to identify dermis and DAPI (dots) to identify cell nuclei. The dashed line indicates the interface between the top layer of the skin, the epidermis, and the bottom layer of the skin, the dermis. Micropatterning of the PLGA has replicated formation of Rete ridges.

FIG. 6 shows an image of a transverse section of normal human skin. Normal human skin transverse section stained with pan cytokeratin (light grey areas above dashed line) to identify keratinocytes and DAPI (grey dots) to identify cell nuclei. The dashed line indicates the interface between the top layer of the skin, the epidermis, and the bottom layer of the skin, the dermis. This interface is not flat, and is characterised by a wave pattern called Rete ridges.

FIG. 7 Shows an example of a plot obtained from a porometer

### DETAILED DESCRIPTION

The term “matrix”, “cell matrix”, “cellular matrix”, “substrate” or “cell substrate” as used interchangeably herein refers to any physical structure including but not limited to, a solid or semi-solid structure, such as a meshwork of fibres with pores suitable for providing:
mechanical or other support for the adherence and proliferation of cells or tissue, and
allowing migration of the one cell types during the culturing process, for example for ex vivo skin tissue culture.

In contact with the gas permeable layer/membrane as employed herein refers to the relevant cells being on the membrane/layer or in the proximity of the membrane/layer, such that the growth and/or in particular differentiation of the cells can occur. Thus proximity will generally mean that there is nothing separating the gas permeable layer/membrane and the relevant cells (the space therebetween will be filled for example with culture media, buffer or CO₂, in particular cell culture media). In one embodiment the distance of the relevant cells to the gas permeable layer is 2 cm or less, such as 1 cm or less, in particular 0.5, 0.4, 0.3, 0.2, 0.1 or 0.05 cm. In one embodiment the outer of cells for differentiation rests on the gas permeable layer/membrane.

Generally, the matrix will be three dimensional, with a first 2D and second face 2D (with a significant surface area) on which cells may deposited and a depth between the two faces giving the 3 dimension (corresponding to a cross-section of the final skin—somewhere in the region of a 100 μm as discussed above).

The matrices of the present disclosure may be constructed of natural or synthetic materials. A matrix may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

In one embodiment, the matrices comprise only synthetic materials. In another embodiment the matrix comprises a mixture of synthetic and natural materials.

In one embodiment, synthetic materials for making the matrix of the present invention are both biocompatible and biodegradable (e.g. subject to enzymatic and hydrolytic degradation), such as biodegradable polymers.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

The term “biodegradable” or “bioabsorbable” as used herein is intended to describe materials that exist for a limited time in a biological environment and degrade under physiological conditions to form a product that can be metabolized or excreted without damage to the subject. In certain embodiments, the product is metabolized or excreted without permanent damage to the subject.

In one embodiment, the matrix is completely resorbable by the body of a subject. In one embodiment, a bioabsorbable matrix of the present disclosure may exist for days, weeks or months when placed in the context of a biological environment. For example, a bioabsorbable matrix may exist for 1, 2, 3, 4, 5, 6, 7, 8, 9 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180 days or more when placed in the context of biological environment.

In one embodiment, the matrix layer is resorbed by the body of said subject at about a same rate as growth of tissue cells underlying said membrane matrix layer in said area. In certain embodiments, the cells are epithelial cells. In certain embodiments, the matrix layer is substantially completely resorbed by said body within about 3 to 12 months after the skin graft is applied. In certain embodiments, the matrix is substantially completely resorbed within about 3 months.

Biodegradable materials such as polymers may be hydrolytically degradable, may require cellular and/or enzymatic action to fully degrade, for example hydrolysis, oxidation, enzymatic processes, phagocytosis, or other processes, including a combination of the foregoing.

Biodegradable polymers are known to those of ordinary skill in the art and include, but are not limited to, synthetic polymers, natural polymers, blends of synthetic and natural polymers, inorganic materials, and the like.

In one embodiment, the matrix incorporates one or more synthetic polymers in its construction. The matrix may be made from heteropolymers, monopolymers, or combinations thereof. Examples of polymers suitable for manufacturing cell matrices include, but are not limited to aliphatic polyesters, copoly(ether-esters), polyalkylenes oxalates, polyamides, poly(iminocarbonates), polyorthoesters, polyoxaesters, polyamidoesters, polyoxaesters containing amine groups, poly(anhydrides), polyphosphazenes, biomolecules and blends thereof.

Suitable aliphatic polyesters include homopolymers, copolymers (random, block, segmented, tappered blocks, graft, triblock, etc.) having a linear, branched or star structure. Suitable monomers for making aliphatic homopolymers and copolymers may be selected from the group consisting of, but are not limited, to lactic acid, lactide (including L-, D-, meso and D,L mixtures), glycolic acid, glycolide, .epsilon.-caprolactone, p-dioxanone (1,4-dioxan-2-one), trimethylene carbonate (1,3-dioxan-2-one), delta-valerolactone, beta-butyrolactone, epsilon-decalactone, 2,5-diketomorpholine pivalolactone, alpha, alpha-diethylpropiolactone, ethylene carbonate, ethylene oxalate, 3-methyl-1,4-dioxane-2,5-dione, 3,3-diethyl-1,4-dioxan-2,5-dione, gamma-butyrolactone, 1,4-dioxepan-2-one, 1,5-dioxepan-2-one, 6,6-dimethyl-dioxepan-2-one, 6,8-dioxabicycloctane-7-one and combinations thereof.

Elastomeric copolymers also are particularly useful in the presently disclosed matrices. Suitable bioabsorbable biocompatible elastomers include but are not limited to those selected from the group consisting of elastomeric copolymers of epsilon-caprolactone and glycolide (for example having a mole ratio of epsilon-caprolactone to glycolide from about 35:65 to about 65:35, more such as from 45:55 to 35:65) elastomeric copolymers of .epsilon.-caprolactone and lactide, including L-lactide, D-lactide blends thereof or tactic acid copolymers (for example having a mole ratio of epsiton-caprolactone to lactide of from about 35:65 to about 65:35, such as from 45:55 to 30:70 or from about 95:5 to about 85:15) elastomeric copolymers of p-dioxanone (1,4-dioxan-2-one) and lactide including L-lactide, D-lactide and lactic acid (for example having a mole ratio of p-dioxanone to lactide of from about 40:60 to about 60:40) elastomeric copolymers of epsilon-caprolactone and p-dioxanone (for example having a mole ratio of epsilon-caprolactone to p-dioxanone of from about from 30:70 to about 70:30) elastomeric copolymers of p-dioxanone and trimethylene carbonate (for example having a mole ratio of p-dioxanone to trimethylene carbonate of from about 30:70 to about 70:30), elastomeric copolymers of trimethylene carbonate and glycolide (for example having a mole ratio of trimethylene carbonate to glycolide of from about 30:70 to about 70:30), elastomeric copolymer of trimethylene carbonate and lactide including L-lactide, D-lactide, blends thereof or lactic acid copolymers (for example having a mole ratio of trimethylene carbonate to lactide of from about 30:70 to about 70:30) and blends thereof. Examples of suitable bioabsorbable elastomers are described in U.S. Pat. Nos. 4,045,418; 4,057,537 and 5,468,253 all hereby incorporated by reference. These elastomeric polymers will have an inherent viscosity of from about 1.2 dL/g to about 4 dL/g, preferably an inherent viscosity of from about 1.2 dL/g to about 2 dL/g and most preferably an inherent viscosity of from about 1.4 dL/g to about 2 dL/g as determined at 25° C. in a 0.1 gram per deciliter (g/dL) solution of polymer in hexafluoroisopropanol (HFIP).

Other materials suitable for use as a matrix of the present disclosure include, but are not limited to, polylactic acid-glycolic acid (PLGA), polyorthoesters, polyanhydrides, polyphosphazenes, and combinations thereof. Non-biodegradable polymers include polyacrylates, polymethacrylates, ethylene vinyl acetate, polyvinyl alcohols, polylactide, chondroitin sulfate (a proteoglycan component), polyesters, polyethylene glycols, polycarbonates, polyvinyl alcohols, polyacrylamides, polyamides, polyacrylates, polyesters, polyetheresters, polymethacrylates, polyurethanes, polycaprotactone, polyphophazenes, polyorthoesters, polyglycolide, copolymers of lysine and lactic acid, copolymers of lysine-RGD and lactic acid, and the like, and copolymers of the same. Synthetic polymers can further include those selected from the group consisting of aliphatic polyesters, poly(amino acids), poly(propylene fumarate), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, and blends thereof.

In one embodiment, the matrix incorporates polylactic acid (PLA). PLA is particularly suited to tissue engineering methods using the cellular matrix as PLA degrades within the human body to form lactic acid, a naturally occurring chemical which is easily removed from the body. The cellular matrix of the invention may also incorporate polyglycolic acid (PGA) and/or polycaprolactone (PCL) as matrix materials. PGA and PCL have similar degradation pathways to PLA, but PGA degrades in the body more quickly than PLA, while PCL has a slower degradation rate than PLA.

PGA has been widely used in tissue engineering. PGA matrices can be easily manipulated into various three dimensional structures, and offer an excellent means of support and transportation for cells (Christenson L, Mikos A G, Gibbons D F, et al: Biomaterials for tissue engineering: summary. Tissue Eng. 3 (1): 71-73; discussion 73-76, 1997) matrices manufactured from polyglycolic acid alone, as well as combinations of PGA and other natural and/or synthetic biocompatible materials, are within the scope of the present disclosure.

In one embodiment, the matrix comprises poly(lactic-co-glycolic acid) (PLGA), such as PLGA microfiber or nanofibres.

In another embodiment, the matrix comprises dioxanone linear homopolymer, such as 100 dioxanone linear homopolymer (e.g. Dioxaprene 100M).

In one embodiment, the matrix comprises a combination of PLGA and 100 Dioxanone.

The term “fibre” is used herein to refer to materials that are in the form of continuous filaments or discrete elongated pieces of material, typically comprising or composed of biodegradeable polymers such as those described above. The fibres of the present disclosure typically have diameters in the micrometer range, such as 0.5 μm to 5 μm, for example 1 μm, 1.5 μm, 2 μm, 2.5 μm, 3 μm, 3.5 μm, 4 μm, 4.5 μm or 5 μm, in particular in the range 1 to 3 μm.

In one embodiment the fibres have a diameter in the range 0.3 μm to 1.5 μm, such as 0.3, 0.4, 0.5, 0.6, 0.7, 0.8, 0.9, 1, 1.1, 1.2, 1.3, 1.4 or 1.5 μm.

The term “fibre matrix” is used herein to refer to the arrangement of fibres into a supporting framework, such as in the form of a sheet of fibres that can then be used to support cells or other additional materials (see also definition of “matrix” above). Various methods are known to the skilled person which can be used to produce suitable fibers, include, but are not limited to, interfacial polymerization and electrospinning.

In one embodiment, a matrix of the present disclosure is formed using electrospinning.

The term “electrospinning” generally refers to techniques that make use of a high-voltage power supply, a spinneret (e.g., a hypodermic needle), and an electrically conductive collector plate (e.g., aluminium foil or stainless steel). To perform the electrospinning process using these materials, an electrospinning liquid (i.e. a melt or solution of the desired materials that will be used to form the fibres) is generally first loaded into a syringe and is then fed at a specific rate set by a syringe pump.

As the liquid is fed by the syringe pump with a sufficiently high voltage, the repulsion between the charges immobilized on the surface of the resulting liquid droplet overcomes the confinement of surface tension and induces the ejection of a liquid jet from the orifice. The charged jet then goes through a whipping and stretching process, and subsequently results in the formation of uniform nanofibers. Further, as the jet is stretched and the solvent is evaporated, the diameters of the fibres can then be continuously reduced to a desired scale, for example micrometers, or even as small as nanometers and, under the influence of an electrical field, the fibres can subsequently be forced to travel towards a grounded collector, onto which they are typically deposited as a non-woven mat. In the context of the present disclosure, due to the high ratio of surface area to volume and the one-dimensional morphology, electrospun fibres can mimic the architecture of the extracellular matrix.

Examples of materials used to produce the nanofibers of the present disclosure are independently selected from those listed in Tables 1 and 2 below.

TABLE 1
Exemplary Materials for producing electrospun
fibres (natural polymers).

| Materials | Solvent | Materials | Solvent |
|---|---|---|---|
| Chitosan | 90% Acetic | Cellulose | NMMO/water |
| | acid | | |
| Gelatin | Formic | Silk | Methanol |
| | acid | fibroin | |
| Gelatin | TFE | Phospholipids | Chloroform/DMF |
| | | (Lecithin) | |
| Collagen Type | HFIP | Fibrinogen | HFIP/10 × minimal |
| I, II, & III | | | essential medium |
| Collagen Type | HFIP | Hemoglobin | TFE |
| I, II, & III | | | |
| Collagen Type | HFIP | Fibrous calf thymus | Water/ethanol |
| I, II, & III | | Na-DNA | |
| Elastin | HFIP | Virus M13 | THF |
| | | viruses | |
| Hyaluronic | DMF/water | | |
| acid | | | |

TABLE 2
Exemplary Materials for producing electrospun
fibres (synthetic polymers).

| Materials | Solvent | Materials | Solvent |
|---|---|---|---|
| PLGA | THF/DMF | PHBV/collagen | HFIP |
| PLA | HFIP | Hyaluronic | DMF/water |
| | | acid/gelatin | |
| PLA | DCM | Collagen/ | TFE/water |
| | | chondroitin | |
| | | sulfate | |
| PLA | DCM/DMF | Collagen/chitosan | HFIP/TFA |
| PLA | DCM/pyridine | Composites | |
| PCL | DCM/methanol | PDLA/HA | Chloroform |
| PHBV | Chloroform/ | PCL/CaCO3 | Chloroform/ |
| | DMF | | methanol |
| PDO | HFIP | PCL/CaCO3 | DCM/DMF |
| PGA | HFIP | PCL/HA | DCM/DMF |
| PLCL | Acetone | PLLA/HA | Chloroform |
| PLCL | DCM | Gelatin/HA | HFIP |
| PLLA-DLA | Chloroform | PCL/collagen/HA | HFIP |
| PEUU | HFIP | Collagen/HA | HFIP |
| Cellulose | Acetic | PDO/ | HFIP |
| acetate | acid/water | elastin | |
| PEG-b-PLA | Chloroform | PLLA/ | 1,4-dioxane/ |
| | | MWNTs/HA | DCM |
| EVOH | 70% propan-2- | Gelatin/siloxane | Acetic acid/ethyl |
| | ol/water | | acetate/water |
| PVA | Water | PLGA/HA | DCM/water |
| PEO | Water | Sodium | Water |
| | | aliginate/PEO | |
| PVP | Ethanol/ | Chitosan/ | Acetic |
| | water | PEO | acid/DMSO |
| PLA/PCL | Chloroform | Chitosan/ | Acetic |
| | | PVA | acid |
| Gelatin/PVA | Formic | Gelatin/ | HFIP |
| | acid | elastin/PLGA | |
| PCL/collagen | HFIP | Silk/PEO | Water |
| | | Silk fibroin/ | Formic |
| | | chitosan | acid |

In one embodiment, the matrix of the present disclosure is composed of synthetic microfibers or nanofibres, for example using the materials listed in Table 2.

The selection of a particular polymer and its use in a specified amount or concentration, or range thereof, provides the ability to control, customize and tailor the degradation rate of the polymer and therefore, the degradation rate of the matrix. This is useful because it is desirable for the matrix to remain as part of the skin graft in order to provide structural support to the grown skin tissue but to eventually degrade and be bioabsorbed by the patient's body once the patient's own cells have assimilated the skin graft, thereby eliminating the requirement for the matrix to be retrieved from the patient's body later on.

Various blends of polymers, for example selected from the materials listed in Tables 1 and 2, may be used to form the fibres to improve their biocompatibility as well as their mechanical, physical, and chemical properties.

Once the desired microfiber or nanofiber matrices have been produced, in one embodiment two or more fibre matrices of the present disclosure are layered together. By layering multiple fibre matrices, the superior and unexpected advantages of each fibre matrix can be combined, and in some cases, result in a synergistic effect.

For example, a first matrix may comprise microwells for receiving one or more relevant cells and/or skin tissue, which is then layered on a second matrix having radially-aligned fibres. In this example, the first matrix can provide the benefit of increasing the repair of damaged skin by providing relevant cells and/or skin tissue whereas the second matrix can provide the benefit of directing and enhancing cell migration from the periphery to the centre of the layered matrices. Layering two or more matrices may also help to enhance the watertight properties of a matrix. The skilled person is able to derive various combinations of two or more different matrices in order to achieve desired properties.

In one embodiment, the matrix of the present disclosure may be further treated via a single procedure or a combination of procedures which reduce the number of microorganisms capable of growing in the matrix under conditions at which the matrix is stored and/or distributed.

In one embodiment, the matrix is sterilised using gamma radiation. In another embodiment, the matrix is sterilised using ethylene oxide (EtO). In another embodiment, the matrix is sterilised using Revox which utilises percetic acid.

In one embodiment, the matrix is sterilized using ionizing radiation such as E-beam irradiation. Electron beam processing has the shortest process cycle of any currently recognized sterilization method. E-beam irradiation, products are exposed to radiation for seconds, with the bulk of the processing time consumed in transporting products into and out of the radiation shielding. Overall process time, including transport time, is 5 to 7 minutes. Electron beam processing involves the use of high energy electrons, typically with energies ranging from 3 to 10 million electron volts (MeV), for the radiation of single use disposable medical products. The electrons are generated by accelerators that operate in both a pulse and continuous beam mode. These high energy levels are required to penetrate product that is packaged in its final shipping container. As the beam is scanned through the product, the electrons interact with materials and create secondary energetic species, such as electrons, ion pairs, and free radicals. These secondary energetic species are responsible for the inactivation of the microorganisms as they disrupt the DNA chain of the microorganism, thus rendering the product sterile. The skilled addressee is aware of other possible methods for sterilising the matrices of the present disclosure.

### Cells for Seeding the Matrices of the Present Disclosure

The matrices of the present disclosure are suitable for supporting the growth of various cells types, for example epithelial cells and/or epidermal cells.

The terms “epithelia” and “epithelium” refer to the cellular covering of internal and external body surfaces (cutaneous, mucous and serous), including the glands and other structures derived therefrom, e.g., corneal, esophageal, laryngeal, epidermal, hair follicle and urethral epithelial cells.

In one embodiment, the epithelial cells employed are skin cells, such as human skin cells, for example cells which form an epidermis and dermis, such as fibroblasts and keratinocytes.

Other exemplary epithelial tissues include: olfactory epithelium, which is the pseudostratified epithelium lining the olfactory region of the nasal cavity, and containing the receptors for the sense of smell; glandular epithelium, which refers to epithelium composed of secreting cells; squamous epithelium, which refers to epithelium composed of flattened plate-like cells.

As used herein “fibroblasts” are understood to be naturally occurring fibroblasts, more particularly fibroblasts occurring in the dermis, genetically modified fibroblasts or fibroblasts emanating from spontaneous mutations or precursors thereof.

As used herein “keratinocytes” are understood to be cells of the epidermis which form keratinizing plate epithelium, genetically modified keratinocytes or keratinocytes emanating from spontaneous mutations or precursors of such keratinocytes which may be of animal or human origin. Alternatively, to the normal skin keratinocytes, mucous membrane keratinocytes or intestinal epithelial cells may be applied to the matrix. These are for example pre-cultivated cells and, in one embodiment, keratinocytes in the first or in the second cell passage, although cells from higher passages may also be used.

The fibroblasts and keratinocytes are obtained and cultivated by methods known among skilled addressees which may be adapted to the required properties of the skin tissue to be produced.

In one embodiment other cell types and/or other cells of other tissue types of both human and animal origin, for example, of mammals, and/or precursor cells thereof, for example, melanocytes, macrophages, monocytes, leukocytes, plasma cells, neuronal cells, adipocytes, induced and non-induced precursor cells of Langerhans cells, Langerhans cells and other immune cells, endothelial cells, cells from tumors of the skin or skin-associated cells, more particularly sebocytes or sebaceous gland tissue or sebaceous gland explantates, cells of the sweat glands or sweat gland tissue or sweat gland explantates, hair follicle cells or hair follicle explantates; and cells from tumors of other organs or from metastases, may be sown on the matrix before, during or after sowing of the keratinocytes. The cells mentioned may be of human and/or animal origin (such as human origin). Stem cells of various origins, tissue-specific stem cells, embryonal and/or adult stem cells may also be incorporated in the skin model.

Accordingly, the process and matrix according to the present disclosure is capable of generating full thickness human skin, which is made up of two tissue-specific layers, namely a dermis equivalent and an epidermis equivalent. The skin tissue substantially corresponds to native skin both histologically and functionally.

The term “tissue” is used to refer to an aggregation of similarly specialized cells united in the performance of a particular function. Tissue is intended to encompass all types of biological tissue including both hard and soft tissue. A “tissue” is a collection or aggregation of particular cells embedded within its natural matrix, wherein the natural matrix is produced by the particular living cells. The term may also refer to ex vivo aggregations of similarly specialized cells which are expanded in vitro such as in artificial organs.

The term “skin tissue,” or “skin” as used herein, refers to any tissue, including epidermis, dermis and basement membrane tissue, for example full thickness skin.

In one embodiment, the cells to be seeded are autologous, that is derived from the subject's own body. In another embodiment, the allogeneic cells are derived from a genetically dissimilar member of the same species. In some cases, xenogeneic cells derived from a species that is different than the intended recipient may be used.

In one embodiment, the sowing of the skin cells on the matrix takes place in the presence of a physiological solution.

The term “physiological solution” as used herein refers to a solution that is similar or identical to one or more physiological condition or that can change the physiological state of a certain physiological environment. The term “physiological solution” as used herein also refers to a solution that is capable of supporting growth of cells (including, but not limited to, mammalian, vertebrate, and/or other cells).

In one embodiment, a physiological solution comprises a defined culture medium, in which the concentration of each of the medium components is known and/or controlled. Defined media typically contain all the nutrients necessary to support cell growth, including, but not limited to, salts, amino acid, vitamins, lipids, trace elements, and energy sources such a carbohydrates. Non-limiting examples of defined media include DMEM, Basal Media Eagle (BME), Medium 199; F-12 (Ham) Nutrient Mixture; F-IO (Ham) Nutrient Mixture; Minimal Essential Media (MEM), Williams' Media E, and RPMI 1640.

In another embodiment, the culture medium is DMEM (Dulbecco's Modified Eagle Medium), M199, Ham's F12 Medium, or a combination thereof. However, any other cell culture medium which allows the cultivation of fibroblasts may also be used.

In one embodiment, Greens medium is used, which is DMEM:Hams F12 (Life Technologies 31765-035) at a ratio of 3:1.

Fetal calf serum (FCS) is preferably used as the serum, although NCS and serum substitute products are also suitable, while Hepes buffer, for example, is used as the buffer. The pH value of the solution of cell culture medium, buffer and serum is preferably in the range from 6.0 to 8.0, for example, from 6.5 to 7.5 and, more particularly, 7.0.

One of ordinary skill in the art will be aware of other defined media that may be used in accordance with the present invention. In one embodiment, a mixture of one or more defined media is employed.

In one embodiment, the media may contain other factors, for example, hormones, growth factors, adhesion proteins, antibiotics, selection factors, enzymes and enzyme inhibitors and the like. Growth factors for example may help to enhance the proliferation of the seeded cells.

The seeding densities of the cellular matrix may vary and the individual layers of the cell matrix may have the same or different seeding densities. Seeding densities may vary according to the particular application for which the cellular matrix is applied. Seeding densities may also vary according to the cell type that is used in manufacturing the cellular matrix.

The number and concentration of cells seeded into or onto the matrix can be varied by modifying the concentration of cells in suspension, or by modifying the quantity of suspension that is distributed onto a given area or volume of the matrix.

In one embodiment, the seeding density is about 150,000 keratinocytes/cm² or higher such as 200,000, 250,000, 300,000, 350,000, 400,000, 450,000, 500,000, 550,000 or 600,000 keratinocytes/cm².

In one embodiment, the seeding density is about 50,000 fibroblasts/cm² or higher, such as 60,000, 70,000, 80,000, 90,000, 100,000, 110,000, 120,000, 130,000, 140,000, 150,000, 160,000, 170,000, 180,000, 190,000 or 200,000 fibroblasts/cm².

Seeding densities of the individual layers of the matrix will depend on the use for which the matrix is intended. Although one skilled in the art may appreciate particular seeding densities a specific application will require, individual layers of the matrix may be seeded at a variety of seeding densities. One skilled in the art will appreciate that the seeding densities for the individual layers of the matrix may vary according to the use for which the matrix is intended.

Spreading involves the use of an instrument such as a spatula to spread the inoculum across the spongiform matrix. Seeding the matrix by painting is accomplished by dipping a brush into the inoculum, withdrawing it, and wiping the inoculum-laden brush across the matrix. This method suffers the disadvantage that substantial numbers of cells may cling to the brush, and not be applied to the lattice. However, it may nevertheless be useful, especially in situations where it is desired to carefully control the pattern or area of lattice over which the inoculum is distributed.

Seeding the matrix by spraying generally involves forcing the inoculum through any type of nozzle that transforms liquid into small airborne droplets. This embodiment is subject to two constraints. First, it must not subject the cells in solution to shearing forces or pressures that would damage or kill substantial numbers of cells. Second, it should not require that the cellular suspension be mixed with a propellant fluid that is toxic or detrimental to cells or wound beds. A variety of nozzles that are commonly available satisfy both constraints. Such nozzles may be connected in any conventional way to a reservoir that contains an inoculum of epithelial stem cells.

Seeding the matrix by pipetting is accomplished using pipettes, common “eye-droppers,” or other similar devices capable of placing small quantities of the inoculum on the surface of the matrix of the present disclosure. The aqueous liquid will permeate through the porous matrix. The cells in suspension tend to become enmeshed at the surface of the matrix and are thereby retained upon the matrix surface.

According to another embodiment of the invention, an inoculum of cells may be seeded by means of a hypodermic syringe equipped with a hollow needle or other conduit A suspension of cells is administered into the cylinder of the syringe, and the needle is inserted into the matrix. The plunger of the syringe is depressed to eject a quantity of solution out of the cylinder, through the needle, and into the scaffold.

An important advantage of utilizing an aqueous suspension of cells is that it can be used to greatly expand the area of matrix on which an effective inoculum is distributed. This provides two distinct advantages. First, if a very limited amount of intact tissue is available for autografting, then the various suspension methods may be used to dramatically increase the area or volume of a matrix that may be seeded with the limited number of available cells. Second, if a given area or volume of a matrix needs to be seeded with cells, then the amount of intact tissue that needs to be harvested from a donor site may be greatly reduced. The optimal seeding densities for specific applications may be determined through routine experimentation by persons skilled in the art.

Typically, the dimensions of the matrix should be substantially planar and of a thickness that gives seeded cells sufficient access to a nutrient medium. When implanted, the cell matrix must have sufficient access to body fluids for nutrition and waste removal. The thickness of the matrix may be varied by changes in the matrix's porosity. Thus, increases in matrix porosity may permit matrices to take on greater thickness as larger pore sizes improve access to external medium and body fluids.

Accordingly, in one embodiment, the matrix has a thickness of 100 μm or less, for example 100, 90, 80, 70, 60, 50, 40, 30, 20, 10, or 5 μm. By keeping the matrix 100 μm or less in thickness, this allows the seeded keratinocytes to receive nutrients and remove waste by diffusion alone, without requiring a vasculature system in order to survive.

Seeding the layered matrix involves introducing one or more desired cell populations to a selected substrate material, and subsequently joining the materials to create a layered matrix. Alternatively, the matrices may be pre-joined, and the selected population(s) of cells introduced at a selected location. Seeding is distinct from the spontaneous infiltration and migration of cells into the matrix from a wound site when the matrix is placed at the wound site.

In one embodiment the matrices are seeded on at least one surface, for example the so-called upper surface of the matrix is seeded with keratinocytes. In one embodiment the so-called lower surface of the matrix is seeded with fibroblasts. In one embodiment the upper surface of the matrix is seeded with keratinocytes and the lower surface of the matrix is seeded with fibroblasts. In one embodiment the upper surface of the matrix is seeded with keratinocytes and the lower surface of the matrix is not seeded with fibroblasts (the fibroblasts are simply allowed to migrate into the matrix from the cell culture). In one embodiment fibroblasts have migrated into the matrix (for example in a pre-culture step) before the keratinocytes are seeded onto the upper surface of the matrix.

### Various Agents to be Attached or Coated to the Matrices of the Present Disclosure

Various additional materials and/or biological molecules can be attached to the matrices of the present disclosure. The term “attached” includes, but is not limited to, coating, embedding or incorporating by any means the additional materials and/or biological molecules, and attached can refer to incorporating such components on the entire matrix or only a portion thereof.

In one embodiment, cell factors are coated/attached to the matrix of the present disclosure. As used herein, the term “cell factors” refers to substances that are synthesized by living cells (e.g. stem cells) and which produce a beneficial effect in the body (e.g. mammalian or human body). Cell factors include, but are in no way limited to, growth factors, regulatory factors, hormones, enzymes, lymphokines, peptides and combinations thereof. Cell factors may have varying effects including, but not limited to, influencing the growth, proliferation, commitment, and/or differentiation of cells (e.g. stem cells) either in vivo or in vitro.

Some non-limiting examples of cell factors include, but are not limited to, cytokines (e.g. common beta chain, common gamma chain, and IL-6 cytokine families), vascular endothelial growth factor (e.g. VEGF-A, -B, -C, -D, and -E), adrenomedullin, insulin-like growth factor, epidermal growth factor EGF, fibroblast growth factor FGF, autocrin motility factor, GDF, IGF, PDGF, growth differentiation factor 9, erythropoietin, activins, TGF-α, TGF-β, bone morphogenetic proteins (BMPs), Hedgehog molecules, Wnt-related molecules, and combinations thereof.

In one embodiment, a growth factor such as EGF (Epidermal Growth Factor), IGF-I (Insulin-like Growth Factor), a member of Fibroblast Growth Factor family (FGF), Keratinocyte Growth Factor (KGF), PDGF (Platelet-derived Growth Factor AA, AB, BB), TGF-β (Transforming Growth Factor family—β1, β2, β3), CIF (Cartilage Inducing Factor), at least one of BMP's 1-14 (Bone Morphogenic Proteins), Granulocyte-macrophage colony-stimulating factor (GM-CSF), or combinations thereof, which may promote tissue regeneration, can be attached to or coated to the matrices of the present disclosure.

In one embodiment, the growth factor is VEGF. In another embodiment, the growth factor is PDGF. The skilled addressee would be aware of various other materials and biological molecules which may be attached to or used to coat a matrix of the presently-disclosed subject matter, and can be selected for a particular application based on the tissue to which they are to be applied.

In one embodiment, an extracellular matrix protein, such as, fibronectin, laminin, and/or collagen, is further attached to or coated on the matrix. Thus, in one embodiment, the matrix is coated with collagen IV, collagen I, laminin and fibronectin, or a combination thereof. The present inventors have discovered that these proteins help provide a secondary cellular signal which in conjunction with growth at an air liquid interface (or gas permeable membrane), causes proper stratification of skin cells grown using the matrix.

In one embodiment, collagen IV is used. Collagen IV was shown to be particularly effective at producing proper epidermal stratification.

The extracellular matrix proteins may be in the form of full length proteins or peptides thereof, for example synthetic peptides.

In another embodiment, a therapeutic agent is further attached to the matrix. The term “therapeutic agent” as used herein refers to any of a variety of agents that exhibit one or more beneficial therapeutic effects when used in conjunction with methods, matrices and/or skin tissues of the present disclosure. Examples of therapeutic agents that may be used include, without limitation, proteins, peptides, drugs, cytokines, extracellular matrix molecules, and/or growth factors. One of skill in the art will be aware of other suitable and/or advantageous therapeutic agents that may be used in accordance with the present disclosure.

In one embodiment, the therapeutic agent is an anti-inflammatory agent or an antibiotic. Examples of anti-inflammatory agents that can be incorporated into the matrices include, but are not limited to, steroidal anti-inflammatory agents such as betamethasone, triamcinolone dexamethasone, prednisone, mometasone, fluticasone, beclomethasone, flunisolide, and budesonide; and non-steroidal anti-inflammatory agents, such as fenoprofen, flurbiprofen, ibuprofen, ketoprofen, naproxen, oxaprozin, diclofenac, etodolac, indomethacin, ketorolac, nabumetone, sulindac tolmetin meclofenamate, mefenamic acid, piroxicam, and suprofen.

Various antibiotics can also be employed in accordance with the presently-disclosed subject matter. Non-limiting examples include aminoglycosides, such as amikacin, gentamicin, kanamycin, neomycin, netilmicin, paromomycin, streptomycin, or tobramycin; carbapenems, such as ertapenem, imipenem, meropenem; chloramphenicol; fluoroquinolones, such as ciprofloxacin, gatifloxacin, gemifloxacin, grepafloxacin, levofloxacin, lomefloxacin, moxifloxacin, norfloxacin, ofloxacin, sparfloxacin, or trovafloxacin; glycopeptides, such as vancomycin; lincosamides, such as clindamycin; macrolides/ketolides, such as azithromycin, clarithromycin, dirithromycin, erythromycin, or telithromycin; cephalosporins, such as cefadroxil, cefazolin, cephalexin, cephalothin, cephapirin, cephradine, cefaclor, cefamandole, cefonicid, cefotetan, cefoxitin, cefprozil, cefuroxime, loracarbef, cefdinir, cefditoren, cefixime, cefoperazone, cefotaxime, cefpodoxime, ceftazidime, ceftibuten, ceftizoxime, ceftriaxone, or cefepime; monobactams, such as aztreonam; nitroimidazoles, such as metronidazole; oxazolidinones, such as linezolid; penicillins, such as amoxicillin, amoxicillin/clavulanate, ampicillin, ampicillin/sulbactam, bacampicillin, carbenicillin, cloxacillin, dicloxacillin, methicillin, mezlocillin, nafcillin, oxacillin, penicillin G, penicillin V, piperacillin, piperacillin/tazobactam, ticarcillin, or ticarcillin/clavulanate; streptogramins, such as quinupristin/dalfopristin; sulfonamide/folate antagonists, such as sulfamethoxazole/trimethoprim; tetracyclines, such as demeclocycline, doxycycline, minocycline, or tetracycline; azole antifungals, such as clotrimazole, fluconazole, itraconazole, ketoconazole, miconazole, or voriconazole; polyene antifungals, such as amphotericin B or nystatin; echinocandin antifungals, such as caspofungin or micafungin, or other antifungals, such as ciclopirox, flucytosine, griseofulvin, or terbinafine.

In one embodiment, various analgesic and/or anesthetic are attached to or incorporated into the matrices of the presently disclosure. As used herein, the term “analgesic” refers to agents used to relieve pain and, in some embodiments, can be used interchangeably with the term “anti-inflammatory agent” such that the term analgesics can be inclusive of the exemplary anti-inflammatory agents described herein. Exemplary analgesic include, but are not limited to: paracetamol and non-steroidal anti-inflammatory agents, COX-2 inhibitors, and opiates, such as morphine, and morphinomimetics.

As used herein, the term “anesthetic” refers to agents used to cause a reversible loss of sensation in subject and can thereby be used to relieve pain. Exemplary anesthetics that can be used in accordance with the presently-disclosed subject matter include, but are not limited to, local anesthetics, such as procaine, amethocaine, cocaine, lidocaine, prilocaine, bupivicaine, levobupivicaine, ropivacaine, mepivacaine, and dibucaine.

### Uses of Matrix and Skin Tissue of the Present Disclosure

The present disclosure provides for the use of tissue, such as epithelium, epidermis, stratified epithelium, stratified epidermis and dermis, split thickness skin or full thickness skin, prepared using a method described herein, for example using a matrix of the present disclosure, for the treatment of tissue damage in a subject in need thereof.

The term “subject” is used herein to refer to both human and animal subjects but is generally intended to refer to a human patient in need of treatment.

The terms “treatment” or “treating,” as used herein include, but are not limited to, inhibiting the progression of damage to a tissue, arresting the development of damage to a tissue, reducing the severity of damage to a tissue, ameliorating or relieving symptoms associated with damage to a tissue, and repairing, regenerating, and/or causing a regression of damaged tissue or one or more of the symptoms associated with a damaged tissue.

The present disclosure provides to a method of treating tissue damage in a subject in need thereof comprising:
a) providing a skin tissue, such as epithelium, stratified epithelium, epidermis, stratified epidermis, stratified epidermis and dermis, split thickness skin or full thickness skin, which has been grown, for example in a method as herein described, such as a method employing a matrix of the present disclosure,
b) recovering under sterile conditions the tissue from the matrix, and
c) applying the tissue to the patient.

In various embodiments the tissue damage is a wound, a chronic wound, a surgical wound, an ulcer, a non-healing wound, a scar, a surgical scar, a scald or a burn. In various embodiments the burn is a first degree burn, a second degree burn, a third degree burn, a deep dermal burn or a full thickness burn.

In various embodiments the tissue damage is epithelium located on a mucosal surface. In various embodiments the epithelium is located on or in skin, the lungs, the gastrointestinal tract (for example, the oesophagus or mouth), reproductive tract, or the urinary tract (for example, the urethra).

Other examples of treatments include but are not limited to: skin regeneration with nerves & organelles; wound healing, for example promoting/enhancing wound healing, including ulcers such as diabetic ulcers; burn healing; skin regeneration and repair; epidermolysis bulosa; enhance skin quality or appearance; prevention or remediation of skin disorders; diminishment or abolishment of scar tissues; breast skin regeneration (after surgery); cosmetic applications, e.g. anti-aging; dermal regeneration for wrinkles and other skin defects; promotion of hair follicle growth, nerve and other organelle regeneration; healing without scarring, or re-healing to diminish scarring.

In one embodiment, the present disclosure provides a matrix, skin tissue and method useful in the regeneration of damaged, lost and/or degenerated tissue. For example, a matrix, method or skin tissue of the present invention may be employed to initiate, increase, support, promote, and/or direct the regeneration of damaged, lost, and/or degenerated tissue, in particular the regeneration of damaged skin.

“Regeneration”, “Regenerate”, “Regenerative” as used herein refer to any process or quality that initiates, increases, modulates, promotes, supports, and/or directs the growth, regrowth, repair, functionality, patterning, connectivity, strengthening, vitality, and/or the natural wound healing process of weak, damaged, lost, and/or degenerating tissue. These terms can also refer to any process or quality that initiates, increases, modulates, promotes, supports, and/or directs the growth, strengthening, functionality, vitality, toughness, potency, and/or health of weak, tired, and/or normal tissue.

As used herein, the term “wound” is used to refer broadly to injuries to the skin and subcutaneous tissue initiated in different ways (e.g., pressure sores from extended bed rest and wounds induced by trauma) and with varying characteristics. Wounds are generally classified into one of four grades depending on the depth of the wound: Grade I: wounds limited to the epithelium; Grade II: wounds extending into the dermis; Grade III: wounds extending into the subcutaneous tissue; and Grade IV (or full-thickness wounds), which are wounds in which bones are exposed (e.g., a bony pressure point such as the greater trochanter or the sacrum).

As used herein, the term “partial thickness wound” refers to wounds that encompass Grades I-III; e.g., burn wounds, pressure sores, venous stasis ulcers, and diabetic ulcers. As used herein, the term “deep wound” is used to describe to both Grade III and Grade IV wounds.

In one embodiment, there is provided is a skin tissue such as epithelium, epidermis, stratified epithelium, stratified epidermis and dermis, split thickness skin or full thickness skin, prepared using a method described herein for facilitating a skin graft, by covering an area of damaged, injured, wounded, diseased, removed or missing skin tissue of a body of a subject.

As used herein, a “graft” refers to a cell, tissue or organ that is implanted into an individual, typically to replace, correct or otherwise overcome a defect. Thus, a “skin graft” is a skin tissue that may be implanted into an individual, for example sutured to the individual. A graft may further comprise a matrix of the present disclosure, for example wherein the matrix is integrated into the skin graft. The tissue or organ may consist of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following interchangeable terms:

“allograft”, “allogeneic transplant”, “allogeneic implant” and “allogeneic graft”. A “xenograft”, “xenogeneic transplant” or “xenogeneic implant” refers to a graft from one individual to another of a different species.

In one embodiment the tissue is prepared using cells that are autologous to the subject. For example, in various embodiments the tissue is prepared using fibroblasts, keratinocytes, or fibroblasts and keratinocytes that are autologous to the subject. In an alternative embodiment the tissue is prepared using cells that are heterologous to the subject. In a further embodiment the tissue is prepared using a combination of cells, wherein some of the cells are autologous to the subject and some of the cells are heterologous to the subject. It will be appreciated that cells autologous to the subject may be isolated using any method known in the art. For example, autologous cells may be isolated from a skin sample or skin biopsy taken from the subject by digesting the sample tissue and separating fibroblasts and/or keratinocytes from the digested tissue.

In one embodiment the tissue is an autograft, for example, a skin autograft. In various embodiments the tissue is an epidermal autograft, a split thickness skin autograft or a full thickness skin autograft. In another embodiment the tissue is an allogeneic graft.

It will be appreciated that the application of tissue prepared using cells autologous to the patient, such as an autograft, is highly desirable to reduce or prevent immune rejection of the tissue and to reduce the requirement for ongoing immunotherapy or another ancillary treatments.

In one embodiment the tissue further comprises the matrix. In another embodiment the tissue is separated from the matrix before application to the patient.

Generally, the application of tissue to the patient will be by surgery. In one embodiment, recovery under sterile conditions is during or immediately prior to surgery, for example in the surgical suite.

Generally, the application of tissue to the patient will be at or adjacent the site of tissue damage. In various embodiments the tissues is applied to at least partially cover the site of tissue damage or to completely cover the site of tissue damage.

In one embodiment the tissue is applied to temporarily cover the site of tissue damage. In an alternative embodiment the tissue is applied to permanently cover the site of tissue damage.

### Other Non-Medical Uses

The efficacy and safety of topically applied pharmaceutical, nutraceutical or cosmetic products are typically tested using animal skin or live animals, human cadaver skin or synthetic human skin models.

Morphological differences between animal and human skin means that the excised animal skin or live animals for the testing of products is not optimal. Furthermore, there is considerable ethical concern about the use of live animals or animal skins for testing cosmetic products, including bans on such testing in some countries. For these reasons, there is a strong desire to identify alternatives to animal models for the testing of such products.

Inconsistent and highly variable results have been observed when human cadaver skin is used for product testing.

Accordingly, cells or tissues, such as skin tissue prepared using the device or methods described herein are useful for in vitro testing of pharmaceuticals, nutraceuticals or cosmetic products.

In various embodiments cells or tissue prepared using the device or methods described herein are used to test transdermal penetration of a compound, to test the permeation of a compound across the epidermis, dermis or basement membrane, to test the efficacy of an active ingredient for treating or preventing a condition, for example, a skin condition, or to test the toxicity of a compound.

More particularly, the skin tissue produced in accordance with the present disclosure is suitable for testing products, for example, for effectiveness, unwanted side effects, for example, irritation, toxicity and inflammation or allergenic effects, or the compatibility of substances. These substances may be substances intended for potential use as medicaments, for example as dermatics, or substances which are constituents of cosmetics or even consumer goods which come into contact with the skin, such as laundry detergents, etc.

The skin tissue of the present disclosure may also be used, for example, for studying the absorption, transport and/or penetration of substances. It is also suitable for studying other agents (physical quantities), such as light or heat, radioactivity, sound, electromagnetic radiation, electrical fields, for example, for studying phototoxicity, i.e. the damaging effect of light of different wavelengths on cell structures. The skin tissue may also be used for studying wound healing and is also suitable for studying the effects of gases, aerosols, smoke and dusts on cell structures or the metabolism or gene expression.

In one embodiment the skin tissue of the present disclosure may be employed to study disease mechanisms affecting the skin, such as skin cancer, psoriasis, dermatitis and the like.

In various embodiments the cells or tissue are used to determine if a compound of interest is a skin irritant, for example, to determine if a compound of interest induces a skin rash, inflammation, or contact dermatitis.

The effects of substances or agents on human skin can be determined, for example, from the release of substances, for example, cytokines or mediators, by cells of the human or animal skin model system and the effects on gene expression, metabolism, proliferation, differentiation and reorganization of those cells. Using processes for quantifying cell damage, more particularly using a vital dye, such as a tetrazolium derivative, it is possible, for example, to detect cytotoxic effects on skin cells. The testing of substances or agents using the skin tissue may comprise both histological processes and also immunological and/or molecular-biological processes.

A “test agent” as used herein is any substance that is evaluated for its ability to diagnose, cure, mitigate, treat, or prevent disease in a subject, or is intended to alter the structure or function of the body of a subject Test agents include, but are not limited to, chemical compounds, biologic agents, proteins, peptides, nucleic acids, lipids, polysaccharides, supplements, signals, diagnostic agents and immune modulators. Test agents may further include electromagnetic and/or mechanical forces.

In another embodiment, the skin tissue produced in accordance with the disclosure may be used as a model system for studying skin diseases and for the development of new treatments for skin diseases. For example, cells of patients with a certain genetic or acquired skin disease may be used to establish patient-specific skin model systems which may in turn be used to study and evaluate the effectiveness of certain therapies and/or medicaments.

In one embodiment, the skin tissue may be populated with microorganisms, more particularly pathogenic microorganisms. Population with pathogenic or parasitic microorganisms, including, in particular, human-pathogenic microorganisms.

“Microorganisms” as used herein generally refers to fungi, bacteria and viruses. The microorganisms are preferably selected from fungi or pathogenic and/or parasitic bacteria known to infect skin. These include but are not limited to species of the genus Candida albicans, Trichophyton mentagrophytes, Malassezia furfur and Staphylococcus aureus.

Using a correspondingly populated skin tissue, it is possible to study both the process of a microorganism population, more particularly the infection process, by the microorganism itself and the response of the skin to that population. In addition, the effect of substances applied before, during or after the population on the population itself or on the effects of the population on the skin tissue can be studied.

In various embodiments the cells comprise fibroblasts, keratinocytes or immune cells, or a combination of any two or more thereof. In one embodiment the cells comprise fibroblasts and keratinocytes. In various embodiments the tissue is selected from the group comprising epidermis, stratified epidermis and dermis, stratified epidermis and dermis, split thickness skin or full thickness skin.

Split thickness skin as employed herein refers to epidermis or dermis layer (such as a dermis layer).

In various embodiments the compound is a pharmaceutical compound, a cosmetic compound or a nutraceutical compound.

In various embodiments the compound for testing is applied to tissue alone or in an admixture with pharmaceutically or cosmetically acceptable carriers, excipients or diluents.

In various embodiments the compound for testing is applied topically to the tissue in the form of a sterile cream, gel, pour-on or spot-on formulation, suspension, lotion, ointment, dusting powder, a drench, spray, drug-incorporated dressing, shampoo, collar or skin patch.

“Comprising” in the context of the present specification is intended to mean “including”.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

The background section of the present disclosure includes technically relevant disclosure, which may be employed as a basis for amended of the claims.

The present application claims priority from G1622340.6, incorporated herein in full by reference. Corrections to the present disclosure may be based on the priority disclosure.

The invention will now be described with reference to the following examples, which are merely illustrative and should not in any way be construed as limiting the scope of the present invention.

### EXAMPLES

In Examples 1 to 3, the cells were grown on the matrices of the present disclosure using methods described in Examples 4 to 7.

### Example 1—Determining Optimal Fibre Diameter and Porosity

Fibre diameter and porosity are important to achieving self-organisation of a mixture of fibroblasts and keratinocytes into full thickness skin containing epidermal and dermal layers. Fibre diameter and porosity need to be optimal so that when keratinocytes and fibroblasts are added to the surface the keratinocytes stay on the top and form an epidermis and the fibroblasts are able to migrate into the synthetic matrix to make a dermis. The result is that the skin formed has the synthetic matrix integrated between the dermis and epidermis formed by the fibroblasts and keratinocytes respectively.

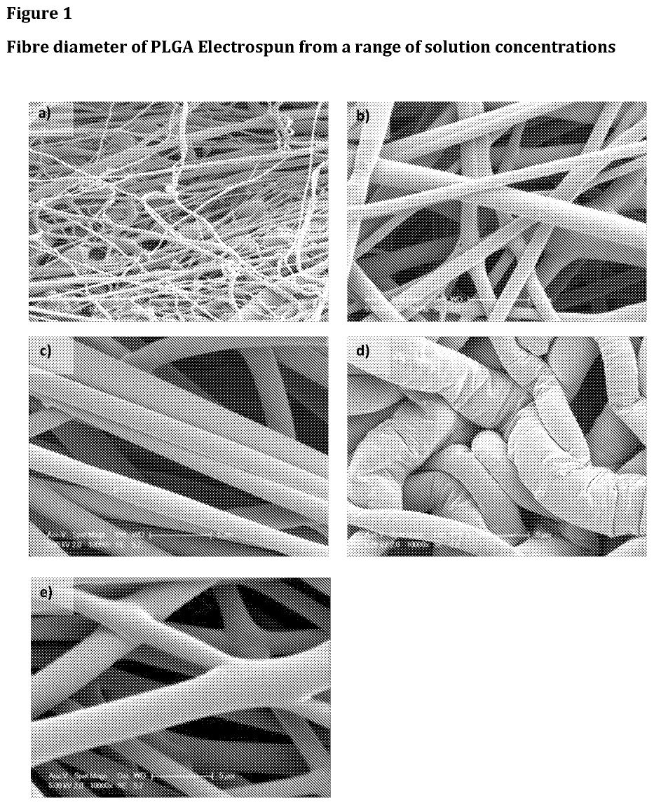

Thus, the present inventors first tested a range of different concentrations of PLGA solution (20% w/v, 25% w/v, 30% w/v, 35% w/v and 40% w/v for electrospinning sheets of fibre. FIG. 1 shows images of the PLGA electrospun fibres. 72:25 PLGA (72:25 ratio of poly-lactic acid to poly-glycolic acid) was used for all the different PLGA concentrations. Electrospinning parameters were as follows:
PLGA dissolved in 1:1 Dimethylformamide:Tetrahydrofuran.
Humidity less than 50%. Temperature 30° C.
Tip of needle on the end of syringe 10 cm away from stainless steel collecting plate.
Flow rate 0.4 ml per hour.
Electrospinning performed for 5 hours. Voltage applied 8 kV.
Stainless steel collector plate contains holes such that the electrospun PLGA mat that forms over the “air” is of lower density compared to the electrospun PLGA mat that forms on the stainless steel collector. The present inventors discovered that low density electrospun PLGA was particularly suitable as a synthetic scaffold for growing full thickness skin.

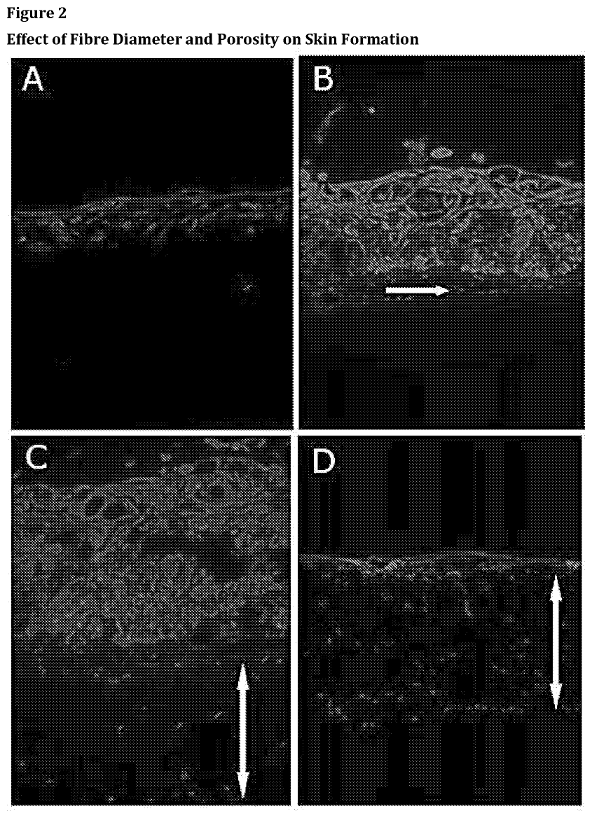

Next, the electrospun PLGA sheets were used as scaffolds for the growth of human skin cells. Using 20% and 25% PLGA solutions resulted in an electrospun PLGA sheets which the fibroblasts were unable to migrate into, they all stayed at the surface (FIGS. 2A and 2B). Using 35% and 40% solutions resulted in electrospun PLGA sheets which the fibroblasts were able to migrate into and keratinocytes stayed on top to form an epidermis (FIGS. 2C and 2D). 35% solution was chosen as optimal because 40% was technically more difficult to work with since it is extremely viscous.

### Example 2—Protein Coating PLGA Matrix and its Effect on Epidermal Stratification

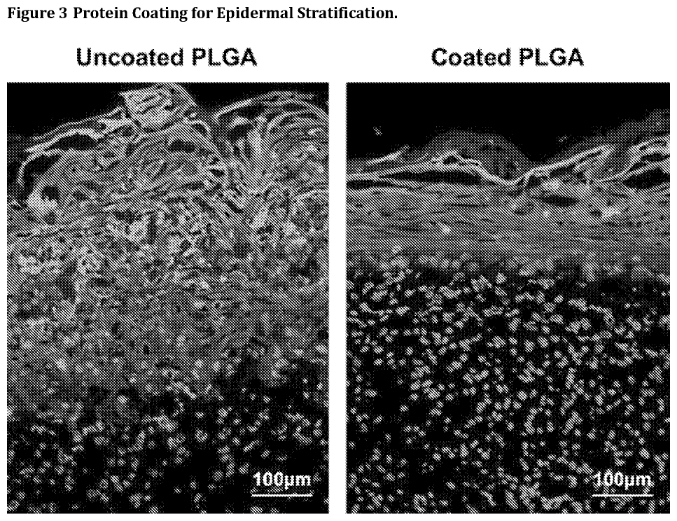

Keratinocytes grown at an air-liquid or gas permeable interface form a stratified epidermis, which is crucial for the barrier function of human skin. Conversely, keratinocytes grown on uncoated electrospun PLGA at an air-liquid or gas permeable interface do not stratify; instead a disorganised epidermis is formed with no stratification. See FIG. 3 uncoated PLGA. A second cellular signal is therefore required in conjunction with the air-liquid or gas permeable interface in order to achieve proper epidermal stratification.

Thus, the present inventors tested a variety of different protein coats on the electrospun PLGA—Collagen IV (10 μg/cm²), Collagen I (10 μg/cm²), Laminin (2 μg/cm²), Fibronectin (5 μg/cm²). All of the coatings were shown to result in proper epidermal stratification when the cells were grown at an air-liquid or gas permeable interface and are all viable alternatives. However, the collagen IV coating consistently produced the best epidermal stratification. See FIG. 3 coated PLGA.

### Example 3—Micropatterning Electrospun PLGA

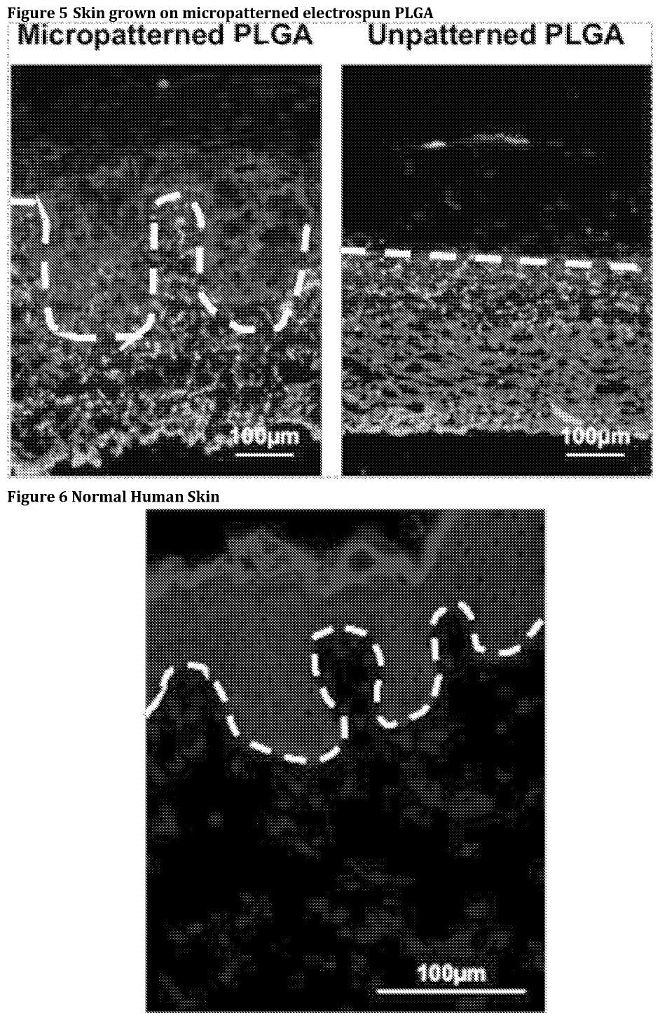

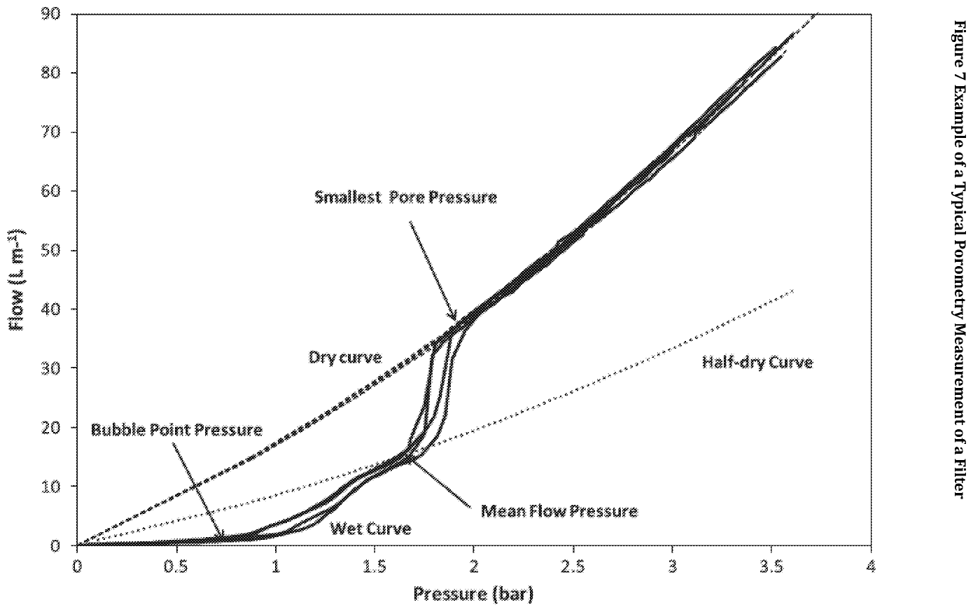

FIG. 6 shows a transverse section of normal human skin. The dashed line indicates the interface between the top layer of the skin, the epidermis, and the bottom layer of the skin, the dermis. This interface is not flat, and is characterised by a wave pattern called Rete ridges. The Rete ridges are contours at the dermal-epidermal interface that provide resistance to shear force and potentially a niche for keratinocyte stem cells.

The present inventors determined that it would be beneficial if cultured skin tissue could replicate these Rete ridges since skin grafts are typically subjected to numerous shear forces for example from a wound dressing or from clothes rubbing on the skin graft.

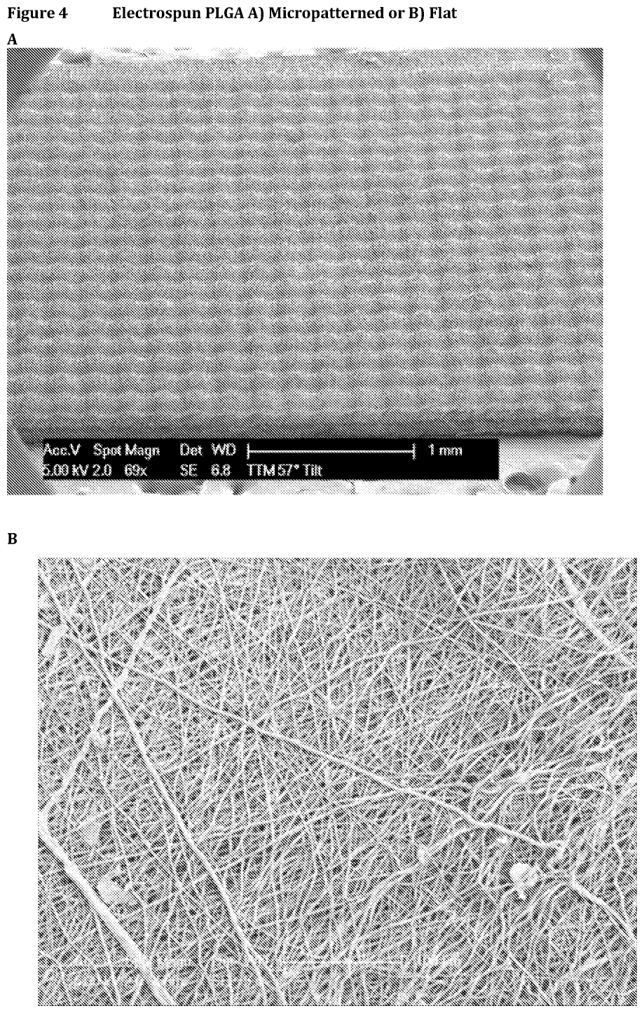

To replicate the Rete ridges, the present inventors developed micropatterned electrospun PLGA sheets. See FIG. 4. The micropatterning of the PLGA was achieved by using a micropatterned collector plate, such as a stainless steel plate.

When the micropatterned PLGA sheets were used as matrices to support the growth of skin tissue, it successfully caused the epidermis and dermis layers to replicate the formation of Rete ridges. See FIG. 3.

### Example 4—Method for Growing Full Thickness Skin Using a Matrix of the Present Disclosure

Sterilised substrate (Electrospun PLGA or any other dermal substitute compatible with skin cell growth) is attached to a stainless steel scaffold. The substrate is optionally coated with collagen IV (Collagen IV Sigma-Aldrich C5533, used at 10 ug/cm²) for 2 hours then washed three times with phosphate buffered saline (PBS).

The scaffold with attached substrate is placed into a gas permeable interface (GPI) apparatus so that the collagen IV-coated side is facing towards the opening.

250 ml of Greens medium (DMEM:Hams F12 (Life Technologies 31765-035) 3:1, 10% FCS, 10 ng/ml EGF (Sigma-Aldrich E9644), 0.4 μg/ml hydrocortisone (Sigma-Aldrich H0396), 0.1 nM choleratoxin (Sigma-Aldrich C8052), 180 μM adenine (Sigma-Aldrich A2786), 5 ug/ml insulin (Sigma-Aldrich 19278), 5 ug/mlapotransferrin (Sigma-Aldrich T2036), 2 nM 3,3,5,-tri-idothyronine (Sigma-Aldrich T2752), 1× Penicillin/Streptomycin, 0.625 μg/ml Amphotercin B (Sigma-Aldrich A2942)) is added to the apparatus.

Fibroblasts and keratinocytes are detached from culture dishes and counted. 300,000 keratinocytes and 100,000 fibroblast per cm² are added into the GPI apparatus. The lid is placed on to seal the GPI apparatus. The apparatus is incubated at 37° C., 5% CO₂ for 48 hours.

The GPI apparatus is inverted, ensuring that the scaffold moves to the opposite end of the apparatus and the substrate is in direct contact with or in proximity the gas permeable membrane. The apparatus is incubated at 37° C., 5% CO₂ for 14 days.

The GPI apparatus is opened, all liquid is discarded, and the scaffold is removed. A scalpel cut around the edges is used to release the skin from the scaffold.

### 2. Result

The method will produce full thickness skin comprising a dermis and stratified epidermis suitable for grafting on to a patient.

### Example 5—Preparation of Full Thickness Skin Using an Apparatus and Method of the Present Disclosure

### 1. Method

Sterilised substrate is attached as described for Example 1 to stainless steel scaffold. The substrate is optionally coated with collagen IV (Collagen IV Sigma-Aldrich C5533, used at 10 ug/cm²) for 2 hours then washed three times with phosphate buffered saline (PBS).

The scaffold with attached substrate is placed into a gas permeable interface (GPI) apparatus so that the collagen IV coated side is facing away from the opening.

250 ml of Greens medium is added as described for Example 4.

100,000 fibroblasts per cm² are added into the GPI apparatus. The lid is placed on the GPI apparatus and sealed. The apparatus is incubated at 37° C., 5% CO₂ for at least 48 hours.

The GPI apparatus is inverted, ensuring that the scaffold moves to the opposite end of the apparatus.

300,000 keratinocytes per cm² are added into the GPI apparatus through the injection port such that the keratinocytes settle on the unseeded side of the substrate. The apparatus is incubated at 37° C., 5% CO₂ for 48 hours.

The GPI apparatus is inverted a second time, ensuring that the scaffold moves to the opposite end of the apparatus and the substrate is in direct contact with the gas permeable membrane. The apparatus is incubated at 37° C., 5% CO₂ for 14 days.

The GPI apparatus is opened, all liquid is discarded, and the scaffold is removed. A scalpel cut around the edges is used to release the skin from the scaffold.

### 2. Result

The method will produce full thickness skin comprising a dermis and stratified epidermis suitable for grafting on to a patient.

### Example 6—Preparation of a Stratified Epidermis Using an Apparatus and Method of the Present Disclosure

### 1. Method

Sterilised substrate is attached as described for Example 4 to stainless steel scaffold. The substrate is optionally coated with collagen IV (Collagen IV Sigma-Aldrich C5533, used at 10 ug/cm²) for 2 hours then washed three times with phosphate buffered saline (PBS).

The scaffold with attached substrate is placed into a gas permeable interface (GPI) apparatus so that the collagen IV coated side is facing towards the opening.

250 ml of Greens medium is added as described for Example 4.

300,000 keratinocytes per cm² are added into the GPI apparatus such that the keratinocytes settle on the unseeded side of the substrate. The lid is placed on the GPI apparatus and sealed. The apparatus is incubated at 37° C., 5% CO₂ for 48 hours.

The GPI apparatus is inverted ensuring that the scaffold moves to the opposite end of the apparatus and the substrate is in contact with the gas permeable membrane. The apparatus is incubated at 37° C., 5% CO₂ for 14 days.

The GPI apparatus is opened, all liquid is discarded, and the scaffold is removed. A scalpel cut around the edges is used to release the stratified epidermis from the scaffold.

### 2. Result

The method will produce a stratified epidermis suitable for grafting on to a patient.

### Example 7—Comparison of Skin Prepared Using a Method of the Present Disclosure Utilising a Gas Permeable Interface (GPI) with Skin Prepared Using a Prior Art Method Utilising an Air-Liquid Interface (ALI)

### 1. Preparation of Full Thickness Skin

### Preparation of Adhered Cells

De-epidermised acellular dermis (DED) was placed in a polystyrene tissue culture dish. A stainless steel ring with a 10 mm diameter aperture and 10 mm depth was set on DED and filled with Green's medium. 300,000 keratinocytes and 100,000 fibroblasts were added into the centre of the ring. The media was changed twice within 24 hours.

### Preparation of Full Thickness Skin Using an Air-Liquid Interface (ALI)

Full thickness skin was prepared using a prior art method utilising an air-liquid interface as follows.

After 48 hours the ring was removed from the DED and the DED comprising adhered fibroblasts and keratinocytes transferred onto a stainless steel rack in a tissue culture dish comprising Greens medium. The rack consisted of a grid of holes, raised 7 mm off the base of the culture dish, through which medium can contact the DED. The level of medium in the culture dish was maintained such that the base of the DED, resting on the metal rack, was in contact with the medium and the top surface of the DED, upon which the keratinocytes and fibroblasts had been seeded, was exposed to air creating an air-liquid interface.

The cells were cultured for 14 days at the air-liquid interface with complete medium changes every two to three days.

### Preparation of Full Thickness Skin Using a Gas Permeable Interface

Full thickness skin was prepared using a gas permeable interface (GPI) as follows.

After 48 hours the ring was removed from the DED and the DED transferred into an apparatus comprising a gas permeable membrane. The DED was placed in the device so that the adhered fibroblasts and keratinocytes were in contact with the gas permeable membrane located at the bottom of the apparatus.

The cells were cultured for 14 days at the gas permeable interface.

After 14 days tissue was harvested for analysis to assess the quality of the skin formed in contact with an air-liquid interface or with a gas permeable membrane.

### 2. Comparison of Full Thickness Skin

The skin produced using a GPI was of a similar thickness and appearance to the skin produced using the ALI.

Samples of each skin were stained with antibodies to: cytokeratin 19, a marker of keratinocyte stem cells; cytokeratin 14; a basal keratinocyte marker; and cytokeratin 10, a suprabasal keratinocyte marker; and examined by fluorescent microscopy. Five μm thick transverse sections of each frozen skin sample were fixed with acetone and blocked with a 0.25% casein solution. Primary antibodies against cytokeratin 10, cytokeratin 14, or cytokeratin 19 in Tris buffered saline (TBS) solution containing 1% foetal bovine serum (FBS) covered the sample sections. Samples were incubated for one hour at room temperature. Samples were washed once with TBS, then three times with rocking for five minutes each time. Secondary antibodies specific for each primary antibody with Alexa 488 dye conjugated, in TBS with 1% FBS containing nuclear stain 4′,6-diamidino-2-phenylindole (DAPI), covered the sample sections. Samples were incubated for 30 minutes at room temperature. Samples were washed once with TBS, then twice with rocking for 15 minutes each time. Samples were covered with Prolong Gold mounting solution and a coverslip placed on top. Images were obtained for all samples of DAPI stain and each Alexa 488 stain using a fluorescent microscope.

Skin grown at the air-liquid interface (ALI) and skin grown at the gas permeable interface (GPI) demonstrated formation of a stratified epidermis.

Many layers of keratinocytes were present in both sample types. Changes in the shape of the nucleus of the keratinocytes in the epidermis, from round in the basal region, to flattened in the upper regions, indicated that a stratified epidermis had formed in skin grown at both the ALI and the GPI.

Skin grown at an ALI or GPI demonstrated expression of cytokeratin 10, a suprabasal keratinocyte marker, in the top layer of the epidermis indicating that a stratum corneum layer had been successfully formed, which in turn indicated that the keratinocyte differentiation and epidermal stratification process had been successfully completed.

Skin grown at an ALI or GPI showed expression of cytokeratin 14, a basal keratinocyte marker, in the layers of keratinocytes below the stratum corneum, indicating these keratinocytes were in a proliferative state, which is required for formation of a stratified epidermis. Skin grown at an ALI and GPI contained keratinocytes that stained positive for cytokeratin 19, a keratinocyte stem cell marker. The presence of keratinocyte stem cells indicates that all of the keratinocyte cell types required for continued renewal of the epidermis were present.

A comparison of skin produced at an ALI with skin grown at a GPI indicates that the GPI may result in a greater number of keratinocyte stem cells present in the epidermis, potentially producing a better stratified epidermis.

This example demonstrates that the method of the invention provides for preparation of full thickness skin having a stratified epidermis and similar features to skin produced using a prior art method.

### Example 8—Preparation of Skin Tissue Using a Method and Apparatus of the Present Disclosure

Skin tissue prepared using (1) an apparatus described herein and (2) a prior art apparatus both utilising a GPI, was compared with skin tissue prepared using (3) a prior art method utilising an ALI.

### 1. Method

Electrospun PLGA was coated with collagen IV solution (10 μg/cm²) for 2 hours at 37° C. to form the substrate. The coated PLGA was washed three times with phosphate buffered saline (PBS) before seeding fibroblasts and keratinocytes onto the coated surface. 100 cm² substrate was used for method (1), 6 cm² for method (2) and 1 cm² for method (3).

### Method (1): Preparation of Skin Using a GPI Apparatus Described Herein

Collagen-coated electrospun PLGA was clamped into the scaffold of a GPI apparatus of the invention such that the coated side was flush with the top surface of the scaffold.

The scaffold was placed in the bottom of the GPI apparatus such that the coated side of the electrospun PLGA faced upwards.

The GPI apparatus was filled with 300 ml of Green's medium. The composition of Green's medium is described in Example 1.

13,000,000 keratinocytes and 2,500,000 fibroblasts were added to the GPI apparatus so that the keratinocytes and fibroblasts could attach to the coated electrospun PLGA.

The lid (comprising a GPI) was placed on the GPI apparatus and the GPI apparatus was sealed.

After 48 hours, the GPI apparatus was inverted to move the scaffold to the opposing end of the apparatus (the lid). In this position, the adhered fibroblasts and keratinocytes were in contact with the GPI in the lid.

The cells were cultured for 14 days and required no medium changes for that period of time.

After 14 days, skin tissue was harvested for analysis.

### Method (2): Preparation of Skin Using a Prior Art GPI Apparatus

A stainless steel ring with a 25 mm diameter aperture and 10 mm depth was set on coated electrospun PLGA inside a 5 cm diameter culture dish and filled with Green's medium. 750,000 keratinocytes and 200,000 fibroblasts were added into the centre of the ring. The media was changed twice within 24 hours.

After 48 hours the ring was removed from the coated electrospun PLGA and the coated electrospun PLGA was transferred from the culture dish into a G-Rex10 apparatus (Wilson Wolf) with 20 mL of Green's medium, such that the adhered fibroblasts and keratinocytes were in contact with the GPI located at the bottom surface of the G-Rex10.

The cells were cultured for 14 days and required no medium changes for that period of time.

After 14 days, skin tissue was harvested for analysis.

### Method (3): Preparation of Skin Using a ALI

A stainless steel ring with a 10 mm diameter aperture and 10 mm depth was set on coated electrospun PLGA inside a six well culture plate and filled with Green's medium.

130,000 keratinocytes and 34,000 fibroblasts were added into the centre of the ring. The media was changed twice within 24 hours.

After 48 hours the ring was removed from the coated electrospun PLGA and the coated electrospun PLGA comprising adhered fibroblasts and keratinocytes was transferred onto a stainless steel rack in a tissue culture dish comprising Green's medium. The rack consisted of a grid of holes, raised 7 mm off the base of the culture dish, through which medium can contact the coated electrospun PLGA. The level of medium in the culture dish was maintained such that the base of the coated electrospun PLGA, resting on the metal rack, was in contact with the medium and the top surface of the coated electrospun PLGA, upon which the keratinocytes and fibroblasts had been seeded, was exposed to air creating an air-liquid interface.

The cells were cultured for 14 days at the ALI with complete medium changes every two to three days.

After 14 days, skin tissue was harvested for analysis.

### Analysis

Samples of each skin were stained with antibodies to pan-cytokeratin, a marker of all keratinocyte cells to assess epidermal quality, and vimentin, a marker of fibroblasts, to assess dermal quality, and examined by fluorescent microscopy.

Five μm thick transverse sections of each frozen skin sample were fixed with acetone and blocked with a 0.25% casein solution. Primary antibodies against pan-cytokeratin, or vimentin in Tris buffered saline (TBS) solution containing 1% foetal bovine serum (FBS) covered the sample sections. Samples were incubated for one hour at room temperature. Samples were washed once with TBS, then three times with rocking for five minutes each time. Secondary antibodies specific for each primary antibody with Alexa 488 dye conjugated, in TBS with 1% FBS containing nuclear stain 4′,6-diamidino-2-phenylindole (DAPI), covered the sample sections. Samples were incubated for 30 minutes at room temperature. Samples were washed once with TBS, then twice with rocking for 15 minutes each time. Samples were covered with Prolong Gold mounting solution and a coverslip placed on top. Images were obtained for all samples of DAPI stain and each Alexa 488 stain using a fluorescent microscope.

### 2. Result

Methods (1), (2) and (3) all produced full thickness skin comprising a dermal layer with a stratified epidermis on top of the dermal layer. The dermal layer was the bottom layer of the skin produced, as evidenced by positive staining for the fibroblast marker Vimentin. The dermal layer was a single cell thick for skin tissue produced by all three methods.

A stratified epidermal layer formed above the dermal layer for skin tissue produced by all three methods. The epidermis comprised many layers of keratinocytes, as evidenced by positive staining for keratinocyte marker pan-cytokeratin. Stratification of the epidermis was observed in the pan-cytokeratin staining of skin samples from the layering of the cytokeratin. Keratinocytes in the basal layer were rounded, becoming flattened out in the intervening layers until a stratum corneum forms the top layer.

Stratification of the epidermis was also demonstrated by the morphology of the keratinocyte cell nuclei, shown by DAPI staining, in the layers of the epidermis. In the basal layers of the epidermis, keratinocyte cell nuclei were rounded, indicating healthy, basal keratinocytes capable of proliferation. Moving up through the epidermal layers the keratinocyte cell nuclei flatten out, indicating they have undergone the differentiation process required to achieve stratification. In the top layer where the keratinocyte cells have completed their differentiation process, the cell nuclei were either very thin or had disappeared completely producing a stratum corneum layer consisting of dead keratinocyte cells.

### Example 9 Porometry Analysis

Full porometry analysis was performed by Porometer (Belgium) the results are shown in the table below. The fluid used was Galpore 16. Fluid tension was 16 dyn/cm. Fluid angle θ. First Bubble Point method was first flow. The bubble point pressure in bars 0.08503 and 0.02002. The bubble point flow in 1/min 0.04977 and 0.004977. Mean flow pore pressure in bars was 0.1419 and 0.1157. Smallest pore pressure in bars 0.2181 and 0.1962. Sample area was 298.6 mm². Gas was air. Temperature was 23° C. Shape factor was 1. Initial pressure in 0 bar. Final pressure 0.5 bar. Wet measurements 100. Dry measurements 25%. Pressure slope 360 s/bar.

| | | Average Fibre | Bubble point | Mean Flow | Smallest |
|---|---|---|---|---|---|
| | Thickness | Diameter | pore size (um) | Pore (MFP) | pore size |
| Scaffold | (um) | (nm) | (Largest pore) | size (um) | (um) |
| UoA 75:25 PLGA | | | 7.53 | 4.51 | 2.94 |
| UoA 75:25 PLGA | | | 31.96 | 5.53 | 3.26 |
| UoA 75:25 PLGA | 10 | 465 | 19.74 | 5.02 | 3.10 |
| average | | | | | |
| Revolution Fibres | | | 5.56 | 2.38 | 1.82 |
| 75:25 PLGA - thick | | | | | |
| area | | | | | |
| Revolution Fibres | | | 8.53 | 2.29 | 1.75 |
| 75:25 PLGA - thick | | | | | |
| area | | | | | |
| Revolution Fibres | | | 5.12 | 2.19 | 1.79 |
| 75:25 PLGA - thick | | | | | |
| area | | | | | |
| Revolution Fibres | 10-22 | 605 | 6.40 | 2.29 | 1.79 |
| 75:25 PLGA - thick | | | | | |
| area average | | | | | |
| Revolution Fibres | | | 7.11 | 2.13 | 1.73 |
| 75:25 PLGA - thin | | | | | |
| area | | | | | |
| Revolution Fibres | | | 11.85 | 2.15 | 1.70 |
| 75:25 PLGA - thin | | | | | |
| area | | | | | |
| Revolution Fibres | | | 6.67 | 2.15 | 1.78 |
| 75:25 PLGA - thin | | | | | |
| area | | | | | |
| Revolution Fibres | 10-22 | 605 | 8.54 | 2.14 | 1.73 |
| 75:25 PLGA - thin | | | | | |
| area average | | | | | |
| SNC 75:25 PLGA 1 | | | 17.05 | 6.65 | 3.76 |
| SNC 75:25 PLGA 1 | | | 15.99 | 6.74 | 3.74 |
| SNC 75:25 PLGA 1 | 23 | 1141 | 16.52 | 6.69 | 3.75 |
| average | | | | | |
| SNC 75:25 PLGA 2 | | | 9.69 | 4.00 | 2.64 |
| SNC 75:25 PLGA 2 | | | 9.69 | 4.25 | 2.69 |
| SNC 75:25 PLGA 2 | | | 10.66 | 4.14 | 2.73 |
| SNC 75:25 PLGA 2 | 21 | 618 | 10.02 | 4.13 | 2.69 |
| average | | | | | |
| SNC 75:25 PLGA 3 | | | 18.83 | 5.969 | 3.142 |
| SNC 75:25 PLGA 3 | | | 11.85 | 6.081 | 3.19 |
| SNC 75:25 PLGA 3 | | | 12.54 | 6.103 | 3.316 |
| SNC 75:25 PLGA 3 | 56 | 1453 | 14.41 | 6.05 | 3.22 |
| average | | | | | |

1.-31. (canceled)

32. A section of synthetic skin tissue comprising a matrix in the form of a continuous sheet of electrospun fibres for growing differentiated skin tissue prepared by electrospinning a solution of only synthetic biocompatible biodegradable polymer at a flow rate in the range 0.1 to 0.4 mL per hour, wherein:
the electrospun fibres are about 0.5 to 3 μm,
keratinocytes are accumulated on an external face of the matrix forming an epidermis, fibroblasts have migrated into the matrix forming a dermis, and
said matrix is within the dermis.

33. A section of synthetic skin tissue comprising a matrix in the form of a continuous sheet of electrospun fibres for growing differentiated skin tissue prepared by electrospinning a solution of a biocompatible biodegradable polymer selected from the group PLGA, PLA, PCL, PHBV, PDO, PGA, PLCL, PLLA-DLA, PEUU, cellulose-acetate, PEG-b-PLA, EVOH, PVA, PEO, PVP, blended PLA/PCL, gelatin-PVA, PCT/collagen, sodium aliginate/PEO, chitosan/PEO, chitosan/PVA, gelatin/elastin/PLGA, silk/PEO, silk fibroin/chitosan, PDO/elastin, hyaluronic acid/gelatin, PDLA/HA, PLLA/HA, gelatin/HA, gelatin/siloxane, PLLA/MWNTs/HA, PLGA/HA, 100 dioxanone linear homopolyer and combinations of two or more of the same, wherein:
the electrospun fibres are about 0.5 to 3 μm,
keratinocytes are accumulated on the matrix forming an epidermis,
fibroblasts have migrated into the matrix forming a dermis, and
said matrix is within the dermis.

34. A section of synthetic skin tissue according to claim 32 wherein the polymer is poly(lactic-co-glycolic acid) (PLGA).

35. A section of synthetic skin tissue according to claim 32, wherein the concentration of biocompatible biodegradable polymer is selected from 30, 31, 32, 33, 34, 35, 36, 37, 38 and 39% w/v.

36. A section of synthetic skin tissue according to claim 35, wherein the concentration of biocompatible biodegradable polymer is 35% w/v.

37. A section of synthetic skin tissue according to claim 34, wherein the ratio of poly-lactic acid to poly-glycolic acid in the PLGA is in the range 90:10 to 50:50 respectively, such as 85:15, 80:20, 75:25, 70:30, 65:35 or 60:40.

38. A section of synthetic skin tissue according to claim 37, wherein the ratio of poly-lactic acid to poly-glycolic acid is 75:25 to 65:35, respectively, such as 65:35.

39. A section of synthetic skin tissue according to claim 32, wherein a solvent comprising one or more independently selected from chloroform, ethanol, acetic acid HFIP, propan-2-ol, acetic acid, DMSO, DMF, ethyl acetate, 1,4-dioxane, formic acid and water, is employed with the biocompatible biodegradable polymer.

40. A section of synthetic skin tissue according to claim 32, wherein a textured plate, for example micropatterned, such as undulating or dimpled, was employed to collect the electrospun fibres.

41. A section of synthetic skin tissue according to claim 32, wherein the electrospinning was performed at flow rate of 0.4 mL per hour or 0.3 mL per hour.

42. A section of synthetic skin tissue according to claim 32, wherein the matrix has a thickness of 100 μm or less, for example 10 to 100 μm, such as 10, 20, 30, 40, 50, 60, 70, 80, 90, 95 or 100 μm.

43. A section of synthetic skin tissue according to claim 32, wherein the matrix is coated with an extracellular matrix protein or peptide thereof, for example a synthetic peptide (for example to promote cell adhesion and/or differentiation).

44. A section of synthetic skin tissue according to claim 43, wherein the extracellular matrix protein is selected from the group consisting of: collagen IV, collagen I, laminin and fibronectin, and combination of two or more thereof, in particular collagen IV.

45. A section of synthetic skin according to claim 32, wherein pores suitable for allowing migration of fibroblasts, for example the pores are in the range 2 to 30 microns.

46. A section of synthetic skin according to claim 32, wherein the said external surface of the matrix was pre-coated with collagen, such as collagen IV before the addition of the epidermal cells, such as keratinocytes and fibroblasts, to the culture.

47. A section of synthetic skin tissue according to claim 32, wherein the biocompatible biodegradable polymer making up the matrix, has started degrading.

48. A section of synthetic skin tissue according to claim 32, where the skin tissue comprises synthetic Rete ridges.
